SEQUENCE LISTING (7 sequences)

SEQ ID NO: 1 - DNA, 22 bp, Artificial
  Synthetic primer
ttttttttttttttttttttvn

SEQ ID NO: 2 - DNA, 20 bp, Artificial
  Synthetic primer
cagtgatgcctccacctcaa

SEQ ID NO: 3 - DNA, 21 bp, Artificial
  Synthetic primer
tttccctctcccctcagaaat

SEQ ID NO: 4 - DNA, 21 bp, Artificial
  Synthetic primer
ggcggcaccaccatgtaccct

SEQ ID NO: 5 - DNA, 21 bp, Artificial
  Synthetic primer
aggggccggactcgtcatact

SEQ ID NO: 6 - DNA, 20 bp, Artificial
  Synthetic primer
aaccgcattccagagtctgg

SEQ ID NO: 7 - DNA, 23 bp, Artificial
  Synthetic primer
ttcaccacgttgttgtcaagggt

(19) United States
(12) Patent Application Publication
(10) Pub. No.: US 2010/0292316 A1
(43) Pub. Date: Nov. 18, 2010

(54) IMPROVED THERAPEUTIC METHODS AND COMPOSITIONS COMPRISING CHROMAN RING COMPOUNDS

(75) Inventors: Bob G. Sanders, Austin, TX (US); Kimberly Kline, Austin, TX (US)

Correspondence Address: FULBRIGHT & JAWORSKI, L.L.P., 600 CONGRESS AVENUE, SUITE 2400, AUSTIN, TX, 78701, US

(73) Assignee: Research Development Foundation, Carson City, NV (US)

(21) Appl. No.: 12/669,145

(22) PCT Filed: Jul. 9, 2008
(86) PCT No.: PCT/US08/69512

§ 371 Date: Jun. 15, 2010

Related U.S. Application Data
(60) Provisional application No. 60/950,508, filed on Jul. 18, 2007.

(51) Int. Cl. A61K 31/355; A61P 35/00

(52) U.S. Cl. 514/458

(57) ABSTRACT

The instant invention concerns chroman ring derivative compounds such as vitamin E derivatives and methods for their use. In certain aspects, methods for treating subjects comprising Arg, JNK, p73, NOXA or FOXO1 positive cancers are provided. In still further aspects, methods for treating cell proliferative disease such as cancer by administration of a chroman ring compound in conjunction with a P13 or Akt kinase inhibitor are described.

This application claims priority to U.S. Application No. 60/950,508 filed on Jul. 18, 2007, the disclosure of which is specifically incorporated herein by reference in its entirety without disclaimer.

### BACKGROUND OF THE INVENTION

1. Field of the Invention

Methods and compositions for treating subjects with chroman ring compounds such as vitamin E derivatives. In particular methods and compositions for the treatment and prevention of cancer are provided.

2. Description of Related Art

Potent pro-apoptotic vitamin E analogs, such as 2,5,7,8-tetramethyl-2R-(4R,8R,12-trimethyltridecyl)chroman-6-yloxy)acetic acid, referred to as alpha-tocopherol ether analog or α-TEA are promising anticancer therapeutics. In certain synthesis schemes the parent compound for making α-TEA is natural vitamin E (RRR-α-tocopherol) (Lawson et al., 2003). However, derivatives like α-TEA comprise an acetic acid moiety linked to the phenolic oxygen at carbon 6 of the chroman head of RRR-α-tocopherol by an ether linkage yielding a stable, nonhydrolyzable compound (Lawson et al., 2003, Lawson et al., 2004-CCP). α-TEA as well as a number of other chroman derivative compounds have been shown to exhibit anticancer activities in a variety of cancer cell types in culture; as well as, in murine tumor explant models (U.S. Pat. No. 6,703,384; Lawson et al., 2003; Lawson et al., 2004-CCP; Lawson et al., 2004-EBM; Anderson et al., 2004-CR; Anderson et al., 2004-EBM; Zhang et al., 2004). Thus, α-TEA and related compounds appear to be a promising novel chemotherapeutic agent for cancer. Furthermore, α-TEA was shown to have enhanced antitumor efficacy when encapsulated in particals such as liposomes (U.S. Publication 20030236301). However, to date there has been limited information regarding what particular types of cancer cells would be ideal candidates for therapy with chroman ring derivative compounds. Furthermore, little has been known regarding the mechanism of action for chroman ring compounds. Further information regarding a mechanism of action may elucidate new ways to augment anticancer therapies with these compounds.

### SUMMARY OF THE INVENTION

In certain embodiments of the invention there is provided a method for treating cancer patients comprising particular types of cancers. The skilled artisan will recognize that certain types of cancers are more or less susceptible to a given anticancer therapy. In some aspects, cancer cells may be characterized by genes expressed in the cells and thus the susceptibility of a cancer to a given anticancer therapy may be ascertained by determining a gene expression profiled for the cancer. The instant invention provides methods for treating particular types of cancers comprising administering an effective amount of a chroman ring anticancer compound. For example, in certain aspects there is provided a method for treating a cancer patient wherein the patient comprises a cancer cell that is positive for expression of a gene selected from Table 1A or 2A. As used here, the term expression refers to production of an mRNA or polypeptide product from a gene. Thus, in certain aspects, the expression of a given gene in a sample may be determined by detecting an mRNA molecule or a polypeptide encoded by the gene.

Furthermore, in some aspects, there is provided a method for treating a cancer patient wherein the cancer patient comprises a cancer cell comprising reduced expression of a gene selected from Table 1B or 2B. As used herein the term reduced expression references to the level of expression of a mRNA or polypeptide gene product in a cancer cell as compared to a non-cancer cell. Preferably, the expression observed in a cancer cell is compared (directly or indirectly) the expression observed in a normal cell from the same tissue of the cancer cell's origin. Thus, in some aspects, the expression level of a gene may be determined in a cancer and a normal cell. In other aspects, the expression level of a gene in cancer cell may be determined and compared to an expression level from a normal cells as previously ascertained. For example, the expression of a gene in a normal cell may be from a reference database, such as a database comprising average gene expression levels from cells in a particular tissue type.

Thus, in certain specific embodiments of the invention there is provided a method for treating a cancer patient wherein the patient comprises an Arg, JNK (e.g., JNK1 or JNK2), p73, NOXA or FOXO1 positive cancer comprising administering an effective amount of a chroman ring derivative compound. In certain preferred aspects of the invention, an Arg, JNK, p73, NOXA or FOXO1 positive cancer is further defined as a cancer that expresses 2, 3, 4, or more of said genes. As described supra, an Arg, JNK, p73, NOXA or FOXO1 positive cancer may comprise a cancer that expresses an Arg, JNK, p73, NOXA or FOXO1 mRNA or polypeptide. In still further aspects, a patient for treatment according to the invention is further defined as comprising a cancer that does not express constitutively active Akt kinase. Furthermore, in certain aspects, there is provided a method for treating a cancer patient wherein the patient comprises a cancer cell that overexpresses an Arg, JNK, p73, NOXA or FOXO1 gene relative to a normal cell. As detailed above in certain preferred aspects a “normal” cell is defined as a cell that is from the same tissue type as the patient's cancer.

Thus, in yet further aspects of the invention there is provided a method for treating a cancer patient comprising (i) obtaining or having a sample from the patient comprising proteins or nucleic acids from a cancer cell; (ii) determining whether the cancer cell expresses an Arg, JNK, p73, NOXA or FOXO1 gene; and (iii) treating the patient with an effective amount of a chroman ring derivative compound or another anti cancer therapy depending upon whether the cancer cell expresses a Arg, JNK, p73, NOXA or FOXO1 gene. As used herein the term “other” anticancer therapy refers to an anticancer that does not comprise a chroman rinf compound or more specifically does not comprise α-TEA. A sample may be directly obtained from a patient for example via a tumor biopsy or excision of a tumor. However, in certain aspects, a sample may be obtained by a third party such as health care professional for later analysis. Thus, in certain aspects, a sample a may be a frozen or banked patient sample. In a highly preferred embodiment, a sample from a patient will be essentially free from proteins and/or nucleic acids from non-cancer cells. Furthermore, in certain cases, a sample may comprise live cancer cells. Thus, in certain cases, the expression of genes in the cancer cells may be determined after or while the cells are exposed to a compound such as a chroman ring derivative compound. Thus, in certain embodiments, methods of the invention concern determining expression of a gene (e.g., an Arg, JNK, p73, NOXA or FOXO1 gene) in a sample of living cancer cells that have been exposed to a chroman ring derivative compound of the invention.

Methods for determining gene expression in a sample are well known in the art. As described supra, gene expression may be determined by assessment of polypeptide or mRNA expression. For example, a sample comprising a nucleic acid may be analyzed by reverse transcription PCR and/or by nucleic acid hybridization (e.g., labeled probe hybridization) to determine expression of given mRNA such as an an Arg, JNK, p73, NOXA or FOXO1 mRNA. In certain preferred aspects, a sample may be hybridized to an array comprising two or more nucleic acid probes to determine the expression of at least two mRNAs. In still further aspects, a sample comprising a polypeptide may be used to determine expression of a gene in a sample. For example, the expression of a given gene may be assessed by mass spectroscopy or by an antibody binding assay to determine polypeptide expression in a sample. Thus, in certain preferred embodiments, expression of a polypeptide in a sample may be determined by an ELISA or a Western blot analysis.

In certain aspects, methods and compositions of the invention concern the treatment of cancer. For example, in certain cases, a bladder, blood, bone, brain, breast, colon, esophageal, gastrointestinal, gum, head, kidney, liver, lung, nasopharynx, neck, ovary, prostate, skin, stomach, testicular, tongue, or uterine cancer may be treated according to the invention. Furthermore, in certain aspects methods of the invention may comprise administration of one or more additional anticancer therapies such as a chemotherapy, surgical therapy, an immunotherapy or a radiation therapy. Further, specific anticancer therapies for use in the invention as detailed below.

As used herein the terms “chroman ring compound” or “chroman ring derivative” refer to molecules comprising a chroman ring moiety or derivatives thereof. For example, a number of chroman ring derivatives that may be used according to the invention have been previously described in U.S. Pat. Nos. 6,703,384, 6,770,672 and 6,417,223, each incorporated herein by reference. Thus, some specific embodiments, chroman ring compounds and derivatives thereof for us according to the invention include but are not limited to 2,5,7,8-Tetramethyl-(2R-(4R,8R,12-trimethyltridecyl)chroman-6-yloxy)acetic acid (α-TEA), 2,5,7,8-Tetramethyl-(2R-(4R,8R,12-trimethyltridecyl)chroman-6-yloxy)propionic acid, 2.5,7,8-Tetramethyl-(2R-(4R,8R,12-trimethyltridecyl)chroman-6-yloxy)butyric acid, 2,5,8-Trimethyl-(2R-(4R,8R,12-trimethyltridecyl)chroman-6-yloxy)acetic acid, 2,7,8-Trimethyl-(2R-(4R,8R,12-trimethyltridecyl)chroman-6-yloxy)acetic acid, 2,8-Dimethyl-(2R-(4R,8R,12-trimethyltridecyl)chroman-6-yloxy)acetic acid, 2-(N,N-(carboxymethyl)-2(2,5,7,8-tetramethyl-(2R-(4R,8R,12-trimethyltridecyl)chroman-6-yloxy)acetic acid, 2,5,7,8-Tetramethyl-(2RS-(4RS,8RS,12-trimethyltridecyl)chroman-6-yloxy)acetic acid, 2,5,7,8-Tetramethyl-2R-(2RS,6RS,10-trimethylundecyl)chroman-6-yloxy)acetic acid, 3-(2,5,7,8-Tetramethyl-(2R-(4R,8,12-trimethyltridecyl)chroman-6-yloxy)propyl-1-ammonium chloride, 2,5,7,8-Tetramethyl-(2R-(4R,8R,12-trimethyltridecyl)chroman-3-ene-6-yloxy)acetic acid, 2-(2,5,7,8-Tetramethyl-(2R-(4R,8,12-trimethyltridecyl)chroman-6-yloxy)triethylammonium sulfate, 6-(2,5,7,8-Tetramethyl-(2R-(4R,8,12-trimethyltridecyl)chroman)acetic acid, 2,5,7,8-Tetramethyl-(2R-(heptadecyl)chroman-6-yloxy)acetic acid, 2,5,7,8-Tetramethyl-2R-(4,8,-dimethyl-1,3,7 E:Z Nonotrien)chroman-6-yloxy)acetic acid, E.Z,RS,RS,RS-(Phytyltrimethylbenzenethiol-6-yloxy)acetic acid, 1-Aza-.alpha.-tocopherol-6-yloxyl-acetic acid, 1-Aza-N-methyl-.alpha.-tocopherol-6-yloxyl-acetic acid or 2,5,7,8-Tetramethyl-2R-(4,8,12-trimethyl-3,7,11 E:Z tridecatrien)choman-6-yloxy)acetic acid.

In certain aspects, a chroman ring compound may comprise the general structure shown below:

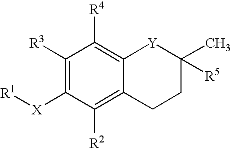

The skilled artisan will recognize that such molecules are related to vitamin E (α-tocopherol). For example, the structure above defines vitamin E when X and Y are each oxygen, R¹ is hydrogen, R², R³ and R⁴ are each methyl and R⁵ is an isopernyl (16 carbon) side chain. Thus, in certain embodiments, a chroman ring compound of the invention may be defined as vitamin E. However, in preferred aspects, a chroman ring compound of the invention may be defined as non-vitamin E chroman ring compound. For instance, some highly preferred chroman ring compounds are defined as having the general structure shown above wherein; X is oxygen; Y is oxygen, N—H or N—CH₃; R², R³, and R⁴ are, independently, hydrogen or methyl; R⁵ is a 16 carbon isopernyl (e.g., a tocopherol) or phytyl (e.g., a tocotrienol) side chain; and R¹ comprises a lower alkyl side chain such as —(CH₂)₁₋₅COOH, —(CH₂)₁₋₅CON(CH₂COOH)₂, —(CH₂)₁₋₅NH₃Cl, —(CH₂)₁₋₅OSO₃NHEt₃, or —(CH₂)₁₋₅COO—(CH₂)₀₋₅CH₃. In certain highly preferred embodiments for instance, R¹ is —(CH₂)₁₋₅COOH, or in particular —CH₂COOH. Thus, in some very specific cases, a chroman ring compound of the invention may be α-TEA (i.e., wherein X and Y are oxygen; R¹ is —CH₂COOH R², R³, and R⁴ are methyl; and R⁵ isopernyl). In still further specific embodiments, a chroman ring molecule may be an α-TEA derivative wherein the isopernyl side chain is substituted for a phytyl side chain.

The skilled artisan will recognize that a chroman ring derivative compound may be administered to a patient by a variety of methods. For example, in preferred, a compound of the invention may be delivered topically, intravenously, orally, or by inhalation. Furthermore, compositions comprising chroman ring derivative compounds of the invention may be encapsulated for example in liposomes as described in U.S. Publn. 20030236301. Further methods for administering compositions of the invention are detailed below.

In some further aspects of the invention there is provided a method for treating a patient with a hyperproliferative disease comprising administering to the patient an effective amount of a chroman ring compound, as described supra, in combination with a Akt and/or PI3K inhibitor. As used herein the term “hyperproliferative disease” comprises cancers and pre cancerous lesions as well as autoimmune disorders resulting from aberrant immune cell proliferation. Thus, in certain very specific aspects there is provided a method for treating a cancer, such as a prostate cancer with an effective amount of a chroman ring compound (e.g., α-TEA) in combination or in conjunction with an Akt and/or PI3K inhibitor. The skilled artisan will recognize that in some aspects chroman ring compounds may be administered before, after or essentially concomitantly with an Akt and/or PI3K inhibitor. Thus, in certain specific aspects, there is provided a medicament composition comprising an effective dose of a chroman ring compound such as α-TEA and an AKT and/or PI3K inhibitor.

A variety of Akt and PI3K inhibitors are know to those in the art. For instance, in some aspects, a PI3K/Akt inhibitors may be SH-5 (A.G. Scientific, Inc., San Diego, Calif.); SH-6 (A.G. Scientific, Inc., San Diego, Calif.), IL-6-hydroxymethyl-chiro-inositol 2(R)-2-O-methyl-3-O-octadecylcarbonate (Martelli et al., 2003), SR13668; wortmannin and LY294002 (Paez & Sellers 2003), API-59 (Tang et al., 2003), KP372-1 (Mandal et al., 2006) or related derivative or prodrug. Additional PI3k/Akt inhibitors can be found in, for example, in U.S. Pat. Nos. 6,245,754, 5,053,399, and 4,988,682 regarding 3-deoxy-D-myo-inositol ether lipid analogs; U.S. Pat. No. 6,187,586 regarding antisense modulation of Akt3 expression; U.S. Pat. No. 6,043,090 regarding antisense inhibition of Akt2 expression; U.S. Pat. No. 5,958,773 regarding antisense modulation of Akt1 expression; and U.S. Pat. No. 6,124,272 regarding antisense modulation of PDK-1 expression. In certain very specific cases a PI3K inhibitor for use according to the invention may be LY294002 (2-(4-morpholinyl)-8-phenyl-4H-1-benzopyran-4-one) or preferably a related quaternary nitrogen prodrug such as one of those described in the U.S. Pat. No. 6,949,537, incorporated herein by reference. For example, in some cases the quaternary nitrogen prodrug is SF1126 (U.S. Pat. No. 6,949,537). Still further PI3 kinase inhibitors that may be used according the invention are described in U.S. Publn. 20030158212 and 20030149074.

In still further embodiments, there is provided a skin care composition comprising a chroman ring derivative such as those described herein and in U.S. Pat. Nos. 6,703,384, 6,770,672 and 6,417,223. Furthermore, in certain aspects, such a skin care composition comprises a liposomal component that enhances the delivery of chroman ring derivatives to the skin. For example certain methods and compositions for lipsome delivery have been described in U.S. Publn. 20030236301. Furthermore, in certain aspects, skin care compositions of the invention may comprise a COX enzyme inhibitor such as celecoxib. Some addition components that may be included in skin care compositions include but are not limited to preservatives, moisturizers, UV blocking agents, emulsifying agents. In a preferred embodiment, skin care compositions of the invention comprise α-TEA. Thus, in certain aspects there are provided sunscreens, tanning oils, moisturizers and sun-less tanning compositions comprising a chroman ring derivative compounds such as α-TEA.

In yet further aspects of the invention there is provided a method for treating or preventing skin lesions (e.g., cancerous or precancerous skin lesions) in a subject by administering a skin care composition comprising a chroman ring derivative. For example, skin care composition comprising chroman ring derivatives such as α-TEA may be applied to the skin before, during or after exposure to UV radiation. Thus, in certain aspects, skin care compositions of the invention may be applied after a session in a of sun tanning or artificial UV tanning (e.g., in a tanning salon) thereby reducing the risk of the appearance of skin lesions.

Embodiments discussed in the context of a methods and/or composition of the invention may be employed with respect to any other method or composition described herein. Thus, an embodiment pertaining to one method or composition may be applied to other methods and compositions of the invention as well.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings are part of the present specification and are included to further demonstrate certain aspects of the present invention. The invention may be better understood by reference to the drawings in combination with the detailed description of specific embodiments presented herein.

FIG. 1A-C: Arg expression is involved in α-TEA induced apoptosis. Arg mRNA and protein expression is up-regulated by α-TEA in MDA-MB-435 but not MCF-7 cells (FIG. 1A-B). MDA-MB-435 and MCF-7 cells were treated with 40 μM of α-TEA or VEH (vehicle control) and harvested at the indicated times. mRNA levels of Arg and β-actin (control) were determined by RT-PCR amplification (FIG. 1A). Protein levels of Arg and PARP (intact and cleavage product p84, an indicator of apoptosis) were determined by Western immunoblotting analyses (FIG. 1B). GAPDH served as a loading control. Data are representative of two independent experiments. FIG. 1C-D, MDA-MB-435 cells were transiently transfected with control siRNA or Arg siRNA, then treated with VEH (vehicle control) or 40 μM of α-TEA for 15 h before analyses for apoptosis using DAPI staining, or Western immunoblot analyses. FIG. 1C, the ability of Arg siRNA to block α-TEA-induced apoptosis. FIG. 1D, Western immunoblot analyses of effects of Arg siRNA on α-TEA-induced Arg protein levels (top panel), and cleavage of PARP (second panel). Data are representative of two independent experiments. Asterisk indicates a statistically significantly difference between the control and Arg siRNA treated cells after α-TEA administration (p<0.005).

FIG. 2A-B: TSP-1 is up-regulated by α-TEA administration. MDA-MB-435, MCF-7 and BALB/c 66c1-4-GFP cells were treated with 40 μM of α-TEA or VEH and harvested at the indicated times. mRNA levels of TSP-1 and β-actin (control) were determined by RT-PCR amplification (FIG. 2A). Protein levels of TSP-1 and PARP cleavage were determined by Western immunoblotting analyses (FIG. 2B). GAPDH serves as a loading control. Data are representative of two independent experiments.

FIG. 3A-B: TSP-1 is not directly involved in α-TEA-induced apoptosis in MDA-MB-435 human breast cancer cells. MDA-MB-435 cells were transiently transfected with control siRNA or TSP-1 siRNA, then treated with VEH (vehicle control) or 40 μM of α-TEA for 15 h before analyses for apoptosis using DAPI staining, or Western immunoblot analyses. FIG. 3A, ability of TSP-1 siRNA to block α-TEA-induced apoptosis. FIG. 3B, Western blot analyses of effects of TSP-1 siRNA on α-TEA-induced TSP-1 protein levels (top panel), and cleavage of PARP (second panel). Data are representative of two independent experiments.

FIG. 4A-B: α-TEA decreased levels of all three phosphorylated active forms of Akt. FIG. 4A, Western Blot analyses of p-Akt, total Akt, p-GSK3β, and total GSK3β in LNCaP and PC-3-GFP cells treated with 40 μM α-TEA for 3, 6, 15, or 24 h, or vehicle for 24 h. GAPDH was used as the loading control. FIG. 4B, Western blot analyses of p-Akt, Akt1, Akt2, and Akt3 in Akt1, Akt2, and Akt3 immunocomplexes immunoprecipitated from LNCaP and PC-3-GFP cells treated with 40 μM α-TEA for 24 h. Data are representative of a minimum of two independent experiments.

FIG. 5A-D: Overexpression of active Akt1 (His/M/Akt1) or Akt2 (HA-Myr-Akt-2) inhibits α-TEA induced apoptosis in LNCap cells. FIG. 5A-B, LNCaP cells transiently transfected with active Akt1 (His/m/Akt1) or Akt2 (HA-Myr-Akt2) are resistant to induction of apoptosis when treated with α-TEA (15 μM for Akt1 or 20 μM for Akt2) induced apoptosis. FIG. 5C-D, Western immunoblot analyses of cell extracts from cells treated in (FIG. A, B) show elevated levels of p-AKT (FIG. C-D, top band), presence of His-Akt1 and HA-Myr-Akt2 (FIG. C-D, second band), and reduced apoptosis as determined by reduced cleavage of PARP (FIG. C-D, third band). GAPDH was used to quantitate the data by densitometric analyses (FIG. C-D, bottom band).

FIG. 6A-B: Downregulation of p-Akt using PI3K inhibitor LY294002 sensitized both LNCaP and PC-3-GFP cells to α-TEA-induced apoptosis. FIG. 6A, Western blot analyses of p-Akt, total Akt, and PARP cleavage in LNCaP and PC-3-GFP cells treated with 10 or 20 μM α-TEA, respectively, in the presence or absence of 10 μM LY294002. GAPDH was used as the loading control. Data are representative of a minimum of two independent experiments. FIG. 6B, LNCaP cells were treated with 40 μM α-TEA and/or 6.25, 12.5 or 25 μM LY294002 for 24 h. PC-3-GFP cells were treated with 40 μM α-TEA and/or 12.5, 25 or 50 μM LY294002 for 24 h. At the end of treatments, adherent and floating cells were collected, washed with PBS, stained with 2 μg/ml DAPI, nuclear morphology examined using fluorescent microscopy and the percentage of apoptotic cells determined. Data represent the average of 3 independent experiments±S.D. (*=P<0.05)

FIG. 7A-C: α-TEA reduced the phosphorylation of FOXO1 by Akt and promoted FOXO1 nuclear localization. FIG. 7A, Western blot analyses of p-FOXO1 and total FOXO1 in LNCaP and PC-3-GFP cells untreated and treated with 40 μM α-TEA for 3, 6, 15, 24 h or vehicle control for 24 h. GAPDH was used as a lane load control. FIG. 7B, Western blot analyses of FOXO1 in cytosolic- and nuclear-enriched fractions of LNCaP and PC-3-GFP cells treated with 40 μM α-TEA for 24 h. GAPDH was used as the cytosolic marker and NUP 98 was used as the nuclear marker to show equal loading and the purity of the cytosolic- and nuclear-enriched fractions. FIG. 7C, Western blot analyses of FlipL and survivin in LNCaP and PC-3-GFP cells treated with 40 μM α-TEA for 3, 6, 15, 24 h or vehicle control for 24 h. GAPDH was used as the loading control. In each case data are representative of a minimum of two independent experiments.

FIG. 8A-D: FOXO1 is involved in α-TEA-induced apoptosis. FIG. 8A, LNCaP cells transiently transfected with FOXO1 siRNA or control siRNA were treated with 40 μM α-TEA for 15 h. Cells were collected and washed with PBS. One aliquot of cells was stained with DAPI and analyzed for apoptosis. FIG. 8C, the remainder of cells were processed and examined by western blot analyses for FOXO1 and PARP cleavage. FIG. 8B, LNCaP cells transiently transfected with wild type Flag-FOXO1 or constitutively active Flag-FOXO1-AAA were treated with 40 μM α-TEA for 15 h. Cells were collected at the end of treatments and washed with PBS. One aliquot of cells were stained with DAPI and analyzed for apoptosis. FIG. 8D, the remainder of cells were monitored by western blot analyses for levels of FOXO1, Flag-FOXO1, Flag-FOXO1-AAA and PARP cleavage. GAPDH was used as a lane load control. Data in FIG. 8A,B represent the average of 3 independent experiments±S.D. (*=P<0.05). Data in FIG. 8C,D are representative of a minimum of two independent experiments.

FIG. 9A-D: The BH3-only protein NOXA is up-regulated in a dose- and time-dependent manner by α-TEA. FIG. 9A,C, MDA-MB-435 and MCF-7 cells were treated with 40 μM of α-TEA for 3, 6, 15 and 24 h or VEH (24 h) and harvested at the indicated times. mRNA level of NOXA were determined by RT-PCR amplification (FIG. 9A). Protein levels of NOXA, and PARP cleavage were determined by Western immunoblotting analyses (FIG. 9C). FIG. 9B,D, MDA-MB-435 and MCF-7 cells were treated with α-TEA at the indicated concentrations for 15 h or 24 h. mRNA level of NOXA were determined by RT-PCR amplification (FIG. 9B). Protein levels of NOXA and PARP cleavage were determined by Western immunoblotting analyses (FIG. 9D). All data are representative of two or more independent experiments.

FIG. 10A-B: NOXA is involved in α-TEA induced mitochondria-dependent apoptotic events in MDA-MB-435 cells. MDA-MB-435 cells were either not transfected or transiently transfected with siRNA specific for NOXA or control siRNA, then treated with 40 μM of α-TEA for 15 h before analyses for apoptosis using DAPI staining, or Western immunoblot analyses. FIG. 10A, the ability of NOXA siRNA to block α-TEA-induced apoptosis. FIG. 10B, Western immunoblot analyses of effects of siRNA to NOXA on α-TEA-induced NOXA protein levels, and cleavage of PARP and levels of cleavage fragments of caspases 9, 3 and 8. Data depicted in FIG. 10A are mean±SD of three independent experiments. Data in FIG. 10B are representative of three independent experiments. (*)=significantly reduced in comparison to cells transfected with control siRNA and treated with α-TEA (p<0.001).

FIG. 11A-D: α-TEA mediates NOXA upregulation through the JNK-signaling pathway. FIG. 11A,B, MDA-MB-435 cells or MCF-7 cells were pretreated with 7.5 μM JNK inhibitor II or DMSO (VEH control) for 2 h, followed by treatment with 40 μM α-TEA for 15 h (435 cells) or 20 h (MCF-7 cells). Western immunoblots of whole cell extracts were performed to determine the effects of pretreating MDA-MB-435 cells or MCF-7 cells with JNK inhibitor II on phosphorylation of JNK substrate c-Jun, FIG. 11A-B (top panel), NOXA expression (second panel) and PARP cleavage (third panel). FIG. 11C-D, MDA-MB-435 cells or MCF-7 cells were transiently transfected with control siRNA or siRNA specific for JNK 1 &2, then treated with ethanol (VEH control) or 40 μM of α-TEA as indicated for 15 h (435 cells) or 20 h (MCF-7 cells). Western immunoblot of whole cell extracts were performed to determine the ability of JNK siRNA to block phosphorylation of JNK1, FIG. 11C-D (top panel), phosphorylation of JNK substrate c-Jun (second panel), full length p73 protein levels (third panel), NOXA protein levels (fourth panel) and PARP cleavage (fifth panel). All data are representative of two or more independent experiments.

FIG. 12A-B: Evidence that α-TEA induces NOXA expression via p73-dependent pathway. MDA-MB-435 cells or MCF-7 cells were transiently transfected with control siRNA or p73 siRNA, then treated with ethanol (VEH control) or 40 μM of α-TEA as indicated for 15 h (435 cells) or 20 h (MCF-7 cells). Western immunoblot of whole cell extracts were performed to determine the ability of p73 siRNA to block full length p73 protein levels, FIG. 12A-B (top panel), NOXA protein levels (second panel) and PARP cleavage (third panel). All data are representative of two or more independent experiments.

FIG. 13: Nomenclature used for naming of various tocopherol and tocotrienol derivatives. Tocopherol and tocotrienol compounds represent examples of a chroman ring compounds. Vitamin E (α-tocopherol) comprises a methyl group at positions R¹, R² and R³ of the chroman ring.

### DETAILED DESCRIPTION OF THE INVENTION

In the studies described here it is demonstrated that (a) α-TEA markedly reduces the phosphorylation level of all endogenously expressed Akt isoforms in both LNCaP (Akt1, -2, and -3) and PC-3 3-GFP (Akt1 and -2) human prostate cancer cells; (b) ectopic overexpression of constitutively active Akt1 or Akt2 significantly inhibited α-TEA-induced apoptosis in both cell lines while inhibition of PI3K, an upstream activator of Akt with the chemical inhibitor LY294002, significantly increased α-TEA-induced apoptosis; (c) analyses of downstream targets of Akt showed decreased levels of phosphorylated forms of GSK313, and the forkhead transcription factor FOXO1 following α-TEA treatments, indicating that α-TEA is indeed reducing Akt kinase activity; (d) ectopic overexpression of wild type or constitutively active FOXO1 significantly enhanced α-TEA-induced apoptosis, while introduction of FOXO1 siRNA into the cells significantly inhibited apoptosis induced by α-TEA. Furthermore, α-TEA treatments promoted the nuclear localization of FOXO1, indicating that α-TEA may be an activator of FOXO1; (e) α-TEA-induced increases in FasL protein expression could be blocked by FOXO1 siRNA and enhanced by ectopic overexpression of constitutively active FOXO1; and (f) α-TEA reduced protein levels of both FlipL and survivin, two pro-survival factors. Taken together, α-TEA is a potent inducer of apoptosis in both androgen-dependent and -independent prostate cancer cells and its pleiotrophic effects include marked downregulation of constitutively expressed phosphorylated Akt, activation of FOXO1 and reduced expression of survival factors FlipL and survivin.

Furthermore, chroman ring compounds of the invention mediated apoptosis in cancer cells by altering expression of a number of genes. These genes are involved in the mechanism of action of chroman ring compounds, thus their expression can be used to determine the susceptibility of a cancer cell to therapies with chroman ring derivatives. Some of the genes identified herein are described below:

ARG: Arg belongs to the Abl family of mammalian nonreceptor tyrosine kinases. Arg does not have a nuclear localization signal (NLS) and DNA-binding domain and thus is localized only in the cytoplasm (Cao et al., 2003). Abl family proteins are involved in cellular responses to stress. Activation of c-Abl by DNA-PK and ataxia telangiectasia mutated gene product in cells exposed to genotoxic agents contributes to DNA damage-induced apoptosis by mechanisms, in part, dependent on p53 and its homolog p73 (Cao et al., 2001). In response to reactive oxygen species (ROS) production, ARG phosphorylates Siva-1 and induces apoptosis by a Siva-1-dependent mechanism. Siva-1 has been shown to induce apoptosis by directly binding to Bcl-xL through its amphipathic domain (Xue et al., 2002). Studies herein show that Arg is up-regulated by α-TEA in estrogen-nonresponsive MDA-MB-435 human breast cancer cells but not in MCF-7 estrogen-responsive cells. Thus, Arg expression in estrogen-nonresponsive cells may be used to determine the effectiveness of tocopherol therapy and guide clinical treatment. Furthermore, these studies may suggests that different signaling pathways are involved in α-TEA treatment in breast cancer cell lines with different estrogen status. Significantly, blockage of Arg using Arg siRNA significantly reduced apoptosis in α-TEA treated MDA-MB-435 human breast cancer cells. Thus, in some aspects, tocophwerol therapies may be enhanced by administration in combination or in conjunction with treatments that up-regulate or activate Arg.

TSP-1: Thrombospondin-1 (TSP-1) belongs to a family of high molecular weight glycoproteins that are secreted by most cell types (Lawler, 2002). Endogenous TSP-1 normally acts to suppress tumor growth in vivo (Sid et al., 2004). The indirect effects of TSP-1 on tumor growth result from its ability to activate TGF-β in the stroma and inhibit activation of matrix metalloproteinases 9, thus resulting in the suppression of tumor cell growth and inhibition of the release of VEGF from the extracellular matrix (Ren et al., 2006). In tumors, TSP-1, that is secreted by stromal cells and some tumor cells, can directly inhibit endothelial cell migration and survival and can stimulate endothelial cell apoptosis, resulting in the down-regulation of angiogenesis and the inhibition of tumor growth (Ren et al., 2006). Here, the inventors show that TSP-1 is up-regulated by α-TEA in both MCF-7 and MDA-MB-435 human breast cancer cells as well as 66c1-4-GFP mouse mammary tumor cells. Since TSP-1 can activate TGF-β in tumor cells and activation of TGF-β signaling pathway is involved in α-TEA-induced apoptosis, we used a TSP-1 siRNA to block TSP-1 and to determine effects on apoptosis. TSP-1 siRNA did not inhibit α-TEA induced apoptosis in MDA-MB-435 cells. In this regard, it is important to note that TSP-1 could have multiple roles not only activating TGF-β signaling pathway in tumor cells but also inducing apoptosis on endothelial cells. Interestingly, α-TEA has been shown to significantly reduce blood vessel density in a preclinical xenograft model transplanted with human MDA-MB-435 breast cancer cells (Zhang et al., 2004). Therefore, α-TEA-induced TSP-1 may induce apoptosis in endothelial cells, thus resulting in inhibition of angiogenesis.

Akt pathway: Akt/protein kinase B is a family of serine/threonine kinases composed of three isoforms: Akt1, Akt2, and Akt3, that plays a major role in survival and can block death receptor Fas-dependent apoptotic signals in human prostate cancer cells (Li et al., 2005; Fresno Vara et al., 2004; new reference Shimada et al., 2004). Typically, Akt is activated by binding of phosphoinositol 3-kinase (PI3K)-generated phosphatidylinositol 3,4,5-trisphosphate following growth factor receptor stimulation, resulting in recruitment of Akt to the cell membrane (Fresno Vaara et al., 2004; Song et al., 2005). Membrane association results in conformational changes inphosphorylation of Akt allowing at residues Thr-308 and Ser-473 to be phosphorylated by upstream kinases 3-phosphoinositide-dependent kinase 1 (PDK1) and PDK2 leads to the activation of Akt (Amaravadi et al., 2005; Li et al., 2005; Fresno Vara et al., 2004). In human prostate cancer, somatic inactivation mutations in the tumor suppressor gene PTEN (phosphatase and tensin homolog deleted on chromosome ten) frequently occur, resulting in constitutively active Akt, which provides prostate cancer cells with a survival advantage (Mulholland et al., 2006; Majumder et al., 2005). Thus, Akt is considered a promising chemotherapeutic target for downregulation in prostate cancer (Li et al., 2005; Hennessy et al., 2005).

FOXO1: Akt has a number of substrates whose phosphorylation by Akt prevents their pro-apoptotic actions, including FOXO1 and glycogen synthase kinase 3 beta (GSK3β). FKHR/FOXO1/FKHR1 (Forkhead in rhabdomyosarcoma) belongs to the Forkhead family of transcription factors and mainly localizes in the nucleus in the absence of Akt activity (Birkenkamp et al., 2003). FOXO1 has been reported to play a role in apoptotic induction by transcriptional upregulation of pro-apoptotic genes such as FasL (Birkenkamp et al., 2003). Phosphorylation of FOXO1 by Akt promotes its nuclear exportation and cytoplasmic retention, thereby inhibiting its transcriptional activity (Birkenkamp et al., 2003; Woods et al., 2002). Akt phosphorylates GSK3β on Ser₉ resulting in inhibition of its kinase activity (Jope et al., 2004). The role of activated GSK3β in prostate cancer cell apoptosis is not known; but GSK3β has been implicated in increasing outer mitochondrial membrane permeability leading to cell death (Pastorino et al., 2005) and GSK3β has been shown to phosphorylate Bax and promote its localization to the mitochondria; thereby promoting cell death (Linseman et al., 2004).

TABLE 1A
Genes upregulated in a α-TEA induced apoptosis

| Gene symbol | Gene name {GenBank Accession No.} |
|---|---|
| FOXO1 | Forkhead box O1 (NM_002015) |
| PMAIP1 | Phorbol-12-myristate-13-acetate-induced protein 1 (APR, NOXA) |
| | (NM_021127; BC013120.1) |
| ABL2 | V-abl Abelson murine leukemia viral oncogene homolog 2 (Arg, Abelson- |
| | related gene) {BC065912.1} |
| TP73 | Tumor protein p73 (NM_005427) |
| JNK-1 | mitogen-activated protein kinase 8 (NM_002750) |
| JNK-2 | Mitogen-activated protein kinase 9 (NM_002752) |
| RND3 | Rho family GTPase 3 {AF498969.1} |
| RET | Ret proto-oncogene (multiple endocrine neoplasia and medullary thyroid |
| | carcinoma 1, Hirschsprung disease) {BC003072.2} |
| ANXA1 | Annexin A1 (BC001275.1) |
| TRIB1 | Tribbles homolog 1 (Drosophila) {AF205437.1} |
| ARHGEF2 | Rho/rac guanine nucleotide exchange factor (GEF) 2 {AB014551.3} |
| RHOB | Ras homolog gene family, member B {AF171089.1} |
| STRN3 | Striatin, calmodulin binding protein 3 {AF243424.1} |
| RPS6KA3 | Ribosomal protein S6 kinase, 90 kDa, polypeptide 3 {AB102662.1} |
| IL1RAP | Interleukin 1 receptor accessory protein {AB006537.1} |
| IER3 | Immediate early response 3 {AF039067.1} |
| STK17A | Serine/threonine kinase 17a (apoptosis-inducing) {AB011420.1} |
| THBS1 | Thrombospondin 1 {AB209912.1} |
| PHLDA1 | Pleckstrin homology-like domain, family A, member 1 {AF220656.1} |
| TNFRSF12A | Tumor necrosis factor receptor superfamily, member 12A {AB035480.1} |
| GADD45B | Growth arrest and DNA-damage-inducible, beta (AF078077.1} |
| SESN2 | Sestrin 2 {AK027896.1} |
| BTG1 | B-cell translocation gene 1, anti-proliferative {BC016759.2} |
| CCNL1 | Cyclin L1 {AF180920.1} |
| SERPINE2 | serine (or cysteine) proteinase inhibitor, clade E (nexin, plasminogen activator |
| | inhibitor type 1), member 2 {BC015663.2} |
| SYTL2 | Synaptotagmin-like 2 {AB046817.1} |
| TES | Testis derived transcript (3 LIM domains) {AF245356.1} |
| JUNB | Jun B proto-oncogene {AK222532.1} |
| MYC | V-myc myelocytomatosis viral oncogene homolog (avian) {BC000141.1} |
| EGR1 | Early growth response 1 {BC073983.1} |
| PBX2 | Pre-B-cell leukemia transcription factor 2 {BC082261.1} |
| NFIL3 | Nuclear factor, interleukin 3 regulated {BC008197.1} |
| MXD1 | MAX dimerization protein 1 {BC069377.1} |
| ATF4 | Activating transcription factor 4 (tax-responsive enhancer element B67) |
| | {BC008090.1} |
| CAMTA2 | Calmodulin binding transcription activator 2 {AB020716.1} |

TABLE 1B
Genes down regulated in a α-TEA induced apoptosis

| Gene | |
|---|---|
| symbol | Gene name {GenBank Accession No.} |
| SNAI2 | Snail homolog 2 (Drosophila) {AK223368.1} |
| NFATC2 | Nuclear factor of activated T-cells, cytoplasmic, |
| | calcineurin-dependent 2 {U43341.1} |
| ST3GAL5 | ST3 beta-galactoside alpha-2,3-sialyltransferase 5 |
| | {AB018356.1} |

### I. Methods for Treatment

In certain aspects the invention concerns methods for treating a cancer patient. For example, in certain cases a patient may comprise a bladder, blood, bone, bone marrow, brain, breast, colon, esophagus, gastrointestine, gum, head, kidney, liver, lung, nasopharynx, neck, ovary, prostate, skin, stomach, testis, tongue, or uterus cancer. Some specific cancer that may be treated according to the invention comprise a: malignant neoplasm; carcinoma; undifferentiated carcinoma; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

The skilled artisan will recognize that a chroman ring derivative compound may be administered to a patient by a variety of methods. For example, a compound of the invention may be delivered topically, intravenously, intradermally, intraarterially, intraperitoneally, intralesionally, intracranially, intraarticularly, intraprostaticaly, intrapleurally, intratracheally, intraocularly, intranasally, intravitreally, intravaginally, intrarectally, intramuscularly, intraperitoneally, subcutaneously, subconjunctival, intravesicularlly, mucosally, intrapericardially, intraumbilically, intraocularally, orally, by inhalation, by injection, by infusion, by continuous infusion, by localized perfusion bathing target cells directly, via a catheter, or via a lavage.

A. Pharmaceutical Preparations

Therapeutic compositions for use in methods of the invention may be formulated into a pharmacologically acceptable format. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains chroman ring derivative will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed., 1990, incorporated herein by reference). A pharmaceutically acceptable carrier is preferably formulated for administration to a human, although in certain embodiments it may be desirable to use a pharmaceutically acceptable carrier that is formulated for administration to a non-human animal, such as a canine, but which would not be acceptable (e.g., due to governmental regulations) for administration to a human. Except insofar as any conventional carrier is incompatible with the active ingredient, its use in the therapeutic or pharmaceutical compositions is contemplated.

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In particular embodiments, the compositions of the present invention are suitable for application to mammalian eyes. For example, the formulation may be a solution, a suspension, or a gel. In some embodiments, the composition is administered via a bioerodible implant, such as an intravitreal implant or an ocular insert, such as an ocular insert designed for placement against a conjunctival surface. In some embodiments, the therapeutic agent coats a medical device or implantable device.

In preferred aspects the formulation of the invention will be applied to the eye in aqueous solution in the form of drops. These drops may be delivered from a single dose ampoule which may preferably be sterile and thus rendering bacteriostatic components of the formulation unnecessary. Alternatively, the drops may be delivered from a multi-dose bottle which may preferably comprise a device which extracts preservative from the formulation as it is delivered, such devices being known in the art.

In other aspects, components of the invention may be delivered to the eye as a concentrated gel or similar vehicle which forms dissolvable inserts that are placed beneath the eyelids.

Furthermore, the therapeutic compositions of the present invention may be administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired. Thus, in some case dosages can be determined by measuring for example changes in serum insulin or glucose levels of a subject.

Precise amounts of the therapeutic composition may also depend on the judgment of the practitioner and are peculiar to each individual. Factors affecting the dose include the physical and clinical state of the patient, the route of administration, the intended goal of treatment (e.g., alleviation of symptoms versus attaining a particular serum insulin or glucose concentration) and the potency, stability and toxicity of the particular therapeutic substance.

B. Additional Therapies

As discussed supra in certain aspects therapeutic methods of the invention may be used in combination or in conjunction with additional anticancer therapies.

Chemotherapy

In certain embodiments of the invention a chroman ring derivative is administered in conjunction with a chemo therapeutic agent. For example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, gemcitabien, navelbine, farnesyl-protein tansferase inhibitors, transplatinum, 5-fluorouracil, vincristin, Velcade, vinblastin and methotrexate, or any analog or derivative variant of the foregoing may used in methods according to the invention.

Radiotherapy

In certain further embodiments of the invention a chroman ring derivative composition may be used in combination or in conjunction with a radiation therapy. Radio therapy may include, for example, y-rays, X-rays, and/or the directed delivery of radioisotopes to tumor cells. In certain instances microwaves and/or UV-irradiation may also used according to methods of the invention. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radio therapeutic agent are delivered to a target cell or are placed in direct juxtaposition with the target cell. To achieve cell killing or stasis, both agents are delivered to a cell in a combined amount effective to kill the cell or prevent it from dividing.

Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B, Her-2/neu, gp240 and p155.

Genes

In yet another embodiment, gene therapy in which a therapeutic polynucleotide is administered before, after, or at the same time as a cell targeting construct of the present invention. Administration of a chroman ring derivative in conjunction with a vector encoding one or more additional gene products may have a combined anti-hyperproliferative effect on target tissues.

Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies. A chroman ring therapy of the invention may be employed alone or in combination with a cytotoxic therapy as neoadjuvant surgical therapy, such as to reduce tumor size prior to resection, or it may be employed as postadjuvant surgical therapy, such as to sterilize a surgical bed following removal of part or all of a tumor.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

Other Agents

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

C. Determination of Gene Expression

It will be understood that in certain aspects the expression of a gene or polypeptide in a sample from a patient will be examined. In certain aspects of the invention, methods for obtaining such as sample are included as part of the invention. However, in other aspects of the invention the proteins for method of the invention may be obtained from sample that have already been collected, such as frozen tissue, blood or biopsy samples or a sample collected by a third party.

### II. Topical Compositions

In some aspects the present invention concerns topical delivery of chroman ring compounds. In certain aspects, chroman ring compounds may be provided in a suitable cosmetic vehicle. Non-limiting examples of suitable cosmetic vehicles include emulsions, creams, lotions, solutions (both aqueous and hydro-alcoholic), anhydrous bases (such as lipsticks and powders), gels, and ointments or by other method or any combination of the forgoing as would be known to one of ordinary skill in the art (Remington's, 1990). Variations and other appropriate vehicles will be apparent to the skilled artisan and are appropriate for use in the present invention.

A. Cosmetic Products

Compositions of the present invention can also be used in many cosmetic products including, but not limited to, sunless skin tanning products, moisturizing creams, sun screens, tanning oils, skin benefit creams and lotions, softeners, day lotions, gels, ointments, foundations, night creams, lipsticks, cleansers, toners, masks, or other known cosmetic products or applications. Additionally, the chroman ring compounds may be formulated as leave-on or rinse-off products.

B. Additional Compounds

Chroman ring compositions of the present invention can include other beneficial agents and compounds such as, for example, acute or chronic moisturizing agents (including, e.g., humectants, occlusive agents, and agents that affect the natural miniaturization mechanisms of the skin), anti-oxidants, sunscreens having UVA and/or UVB protection, emollients, anti-irritants, additional vitamins, trace metals, anti-microbial agents, botanical extracts, fragrances, and/or dyes and color ingredients.

### Moisturizing Agents

Non-limiting examples of moisturizing agents that can be used with compositions and methods of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, acrylates/C10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, beta-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid, sandalwood (santalum album) oil, serine, serum protein, sesame (sesamum indicum) oil, shea butter (butyrospermum parkii), silk powder, sodium chondroitin sulfate, sodium DNA, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

### Antioxidants

Non-limiting examples of antioxidants that can be used with the compositions of the present invention include acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

### Compounds Having UV Light Absorbing Properties

Non-limiting examples of compounds that have ultraviolet light absorbing properties that can be used with the compounds of the present invention include benzophenone, benzophenone-1, benzophenone-2, benzophenone-3, benzophenone-4 benzophenone-5, benzophenone-6, benzophenone-7, benzophenone-8, benzophenone-9, benzophenone-10, benzophenone-11, benzophenone-12, benzyl salicylate, butyl PABA, cinnamate esters, cinoxate, DEA-methoxycinnamate, diisopropyl methyl cinnamate, ethyl dihydroxypropyl PABA, ethyl diisopropylcinnamate, ethyl methoxycinnamate, ethyl PABA, ethyl urocanate, glyceryl octanoate dimethoxycinnamate, glyceryl PABA, glycol salicylate, homosalate, isoamyl p-methoxycinnamate, PABA, PABA esters, Parsol 1789, and isopropylbenzyl salicylate.

### Preservatives

Non-limiting examples of preservatives that may used with compositions of the invention include Phenonip™, and/or any of its constituents phenoxyethanol, methylparaben, butylparaben, ethylparaben, propylparaben, additionally Suttocide®, Germaben™, LiquiPar potassium sorbate, and/or rosemary oleoresin may be used.

### Additional Topical Compounds and Agents

Non-limiting examples of additional compounds and agents that can be used with the compositions of the present invention include, vitamins (e.g. D, E, A, K, and C), trace metals (e.g. zinc, calcium and selenium), anti-irritants (e.g. steroids and non-steroidal anti-inflammatories), botanical extracts (e.g. aloe vera, chamomile, cucumber extract, ginkgo biloba, ginseng, and rosemary), dyes and color ingredients (e.g. D&C blue no. 4, D&C green no. 5, D&C orange no. 4, D&C red no. 17, D&C red no. 33, D&C violet no. 2, D&C yellow no. 10, D&C yellow no. 11 and DEA-cetyl phosphate), emollients (i.e. organic esters, fatty acids, lanolin and its derivatives, plant and animal oils and fats, and di- and triglycerides), antimicrobial agents (e.g., triclosan and ethanol), and fragrances (natural and artificial).

### III. Methods for Producing Antibodies

In certain aspect the instent invention concerns determining polypeptide expression in a sample. The skilled artisan will recognize that a variety of methods for determining exprression employ antibodies that bind to a given polypeptide such as a JNK, p73, NOXA or FOXO1 polypeptide. The following methods exemplify some of the most common antibody production methods.

A. Polyclonal Antibodies

Polyclonal antibodies generally are raised in animals by multiple subcutaneous (sc) or intraperitoneal (ip) injections of the antigen. As used herein the term “antigen” refers to any polypeptide that will be used in the production of a antibodies. Antigens for use according to the instant invention include CRFR2, Ucn 2, Ucn 3, polypeptides or fragments of any of the foregoing. Some very specific examples are the antibodies that bind to Ucn 3, exemplified herein, that may be generating by immunizing an animal with human Gly-Tyr-Ucn 3 that ahs been chemically conjugated to antigenic polypeptide. Furthermore in certain cases, it is preferable to generate antibodies that are selective for a specific CRFR2 protein isoform by using isoform specific polypeptide sequence as the antigen. Thus in certain cases, amino acid sequences according to SEQ ID NO:26, SEQ ID NO:27 or SEQ ID NO:28 may be included in the antigen.

It may be useful to conjugate an antigen or a fragment containing the target amino acid sequence to a protein that is immunogenic in the species to be immunized, e.g. keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glytaraldehyde, succinic anhydride, SOCl₂, or R¹N═C═NR, where R and R¹ are different alkyl groups.

Animals are immunized against the immunogenic conjugates or derivatives by combining 1 mg of 1 μg of conjugate (for rabbits or mice, respectively) with 3 volumes of Freud's complete adjuvant and injecting the solution intradermally at multiple sites. One month later the animals are boosted with ⅕ to 1/10 the original amount of conjugate in Freud's complete adjuvant by subcutaneous injection at multiple sites. 7 to 14 days later the animals are bled and the serum is assayed for specific antibody titer. Animals are boosted until the titer plateaus. Preferably, the animal boosted with the same antigen conjugate, but conjugated to a different protein and/or through a different cross-linking reagent. Conjugates also can be made in recombinant cell culture as protein fusions. Also, aggregating agents such as alum are used to enhance the immune response.

B. Monoclonal Antibodies

Monoclonal antibodies are obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies.

For example, monoclonal antibodies of the invention may be made using the hybridoma method first described by Kohler & Milstein (1975), or may be made by recombinant DNA methods (Cabilly et al.; U.S. Pat. No. 4,816,567).

In the hybridoma method, a mouse or other appropriate host animal, such as hamster is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding 1986).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high level expression of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Culture medium in which hybridoma cells are growing is assayed for production of monoclonal antibodies directed against the target antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson & Pollard (1980).

After hybridoma cells are identified that produce antibodies of the desired specificity, affinity, and/or activity, the clones may be subcloned by limiting dilution procedures and grown by standard methods, Goding (1986). Suitable culture media for this purpose include, for example, Dulbecco's Modified Eagle's Medium or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies of the invention is readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, Morrison et al. (1984), or by covalently joining to the immunoglobulin coding sequence all or part of the coding sequence for a non-immunoglobulin polypeptide. In that manner, “chimeric” or “hybrid” antibodies are prepared that have the binding specificity for any particular antigen described herein.

Typically such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for the target antigen and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

For diagnostic applications, the antibodies of the invention typically will be labeled with a detectable moiety. The detectable moiety can be any one which is capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; biotin; radioactive isotopic labels, such as, e.g., ³H, ¹⁴C, ³²P, ³⁵S, or ¹²⁵I, or an enzyme, such as alkaline phosphatase, beta-galactosidase or horseradish peroxidase.

Any method known in the art for separately conjugating the antibody to the detectable moiety may be employed, including those methods described by Hunter et al. (1962); David et al. (1974); Pain et al. (1981); and Nygren (1982).

The antibodies of the present invention may be employed in any known assay method, such as competitive binding assays, direct and indirect sandwich assays, and immunoprecipitation assays (Zola, 1987).

Competitive binding assays rely on the ability of a labeled standard (which may be a purified target antigen or an immunologically reactive portion thereof) to compete with the test sample analyte for binding with a limited amount of antibody. The amount of antigen in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three part complex. David & Greene, U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

### IV. Pharmaceutical Preparations

Therapeutics for use in methods of the invention may be formulated into a pharmacologically acceptable format. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one non-charged lipid component comprising a siNA, an antibody or a CRFR2 antagonist active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed., 1990, incorporated herein by reference). A pharmaceutically acceptable carrier is preferably formulated for administration to a human, although in certain embodiments it may be desirable to use a pharmaceutically acceptable carrier that is formulated for administration to a non-human animal, such as a canine, but which would not be acceptable (e.g., due to governmental regulations) for administration to a human. Except insofar as any conventional carrier is incompatible with the active ingredient, its use in the therapeutic or pharmaceutical compositions is contemplated.

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

Where clinical application of liposomal compositions containing a siNA (i.e. siNA directed to CRFR2, Ucn 2 or Ucn 3) is undertaken, it will generally be beneficial to prepare the lipid complex as a pharmaceutical composition appropriate for the intended application. This will typically entail preparing a pharmaceutical composition that is essentially free of pyrogens, as well as any other impurities that could be harmful to humans or animals. One may also employ appropriate buffers to render the complex stable and allow for uptake by target cells.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

The therapeutic compositions of the present invention may include classic pharmaceutical preparations. Administration of therapeutic compositions according to the present invention will be via any common route so long as the target tissue is available via that route. For example in the case of antibodies, antibody fragments, or siNA compositions an intravenous route of administration may be preferred. In the case of a small molecule or certain polypeptide inhibitors of CRFR2 signaling routes of administration could additionally include oral routes or even nasal, buccal, rectal, vaginal or topical. Alternatively, administration may be by orthotopic, intradermal subcutaneous, intramuscular, intraperitoneal or intravenous injection. In certain specific cases, compositions according to the current invention maybe administered at there site of actions, such as delivery directly to the skeletal muscle or the pancreas.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired. Thus, in some case dosages can be determined by measuring for example changes in serum insulin or glucose levels of a subject.

Precise amounts of the therapeutic composition may also depend on the judgment of the practitioner and are peculiar to each individual. Factors affecting the dose include the physical and clinical state of the patient, the route of administration, the intended goal of treatment (e.g., alleviation of symptoms versus attaining a particular serum insulin or glucose concentration) and the potency, stability and toxicity of the particular therapeutic substance.

### EXAMPLES

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### The Effect of α-TEA on Gene Expression

In order to identify genes that are involved in α-TEA induced apoptosis in cancer cells the gene expression levels in MDA-MB-435 human breast cancer cells were analyzed after treatment with α-TEA. The MDA-MB-435 cell line is an estrogen-receptor negative/estrogen-nonresponsive epithelial cell line isolated from the pleural effusions of a human with breast cancer (Price et al., 1990). DNA microarray data obtained from treatment of MDA-MB-435 cells with 40 μM α-TEA for 12 h identified approximately 400 genes that gave consistent responses to α-TEA treatment. Genes that passed a minimum quality control threshold were analyzed using online software (The Database for Annotation, Visualization and Integrated Discovery (DAVID)). Genes that are up- or down-regulated by α-TEA in MDA-MB-435 human breast cancer cells treated with 40 μM α-TEA for 12 h are listed in Table 2A-B. Data was obtained from 5 replica microarray experiments and averge values shown.

TABLE 2A-B

| | | | | | |
|---|---|---|---|---|---|
| | | | | | Average |
| CLID | Anot. | Name | Symbol | Accession | level |

Table 2A

| 788232 | 57282 | Sestrin 2 | SESN2 | AA454079 | 2.9795 |
|---|---|---|---|---|---|
| 1892526 | 35293 | Rho family GTPase 3 | RND3 | AI277348 | 2.8326 |
| 110503 | 61708 | La ribonucleoprotein domain family, member 2 | LARP2 | T82817 | 2.812 |
| 753104 | 41664 | Dopachrome tautomerase (dopachrome delta-isomerase, tyrosine-related | DCT | AA478553 | 2.44525 |
| | | protein 2) | | | |
| 160664 | 55432 | Ret proto-oncogene (multiple endocrine neoplasia and medullary thyroid | RET | H24956 | 2.4366 |
| | | carcinoma 1, Hirschsprung disease) | | | |
| 840944 | 55633 | Early growth response 1 | EGR1 | AA486628 | 2.3925 |
| 2125074 | 42341 | Thrombospondin 1 | THBS1 | AI436297 | 2.3486 |
| 667883 | 38367 | Pleckstrin homology-like domain, family A, member 1 | PHLDA1 | AA258396 | 2.2265 |
| 487932 | 56715 | Synaptotagmin-like 2 | SYTL2 | AA045284 | 2.1695 |
| 292515 | 57082 | UDP-N-acteylglucosamine pyrophosphorylase 1 | UAP1 | N68465 | 2.1065 |
| 199367 | 33391 | serine (or cysteine) proteinase inhibitor, clade E (nexin, plasminogen | SERPINE2 | R95691 | 2.07225 |
| | | activator inhibitor type 1), member 2 | | | |
| 898092 | 60389 | Connective tissue growth factor | CTGF | AA598794 | 1.9855 |
| 2556526 | 64016 | Down syndrome critical region gene 1 | DSCR1 | AW073325 | 1.9626 |
| 843070 | 60784 | Nucleoporin 88 kDa | NUP88 | AA488609 | 1.9542 |
| 135791 | 53817 | Tumor necrosis factor receptor superfamily, member 12A | TNFRSF12A | R33355 | 1.9508 |
| 1493527 | 38469 | Asparagine synthetase | ASNS | AA894927 | 1.9295 |
| 882483 | 51366 | Interferon-related developmental regulator 1 | IFRD1 | AA676598 | 1.90775 |
| 1660127 | 53036 | Protein phosphatase 2C, magnesium-dependent, catalytic subunit | PPM2C | AI080633 | 1.90075 |
| 814615 | 51411 | Methylenetetrahydrofolate dehydrogenase (NADP+ dependent) 2, | MTHFD2 | AA480995 | 1.8784 |
| | | methenyltetrahydrofolate cyclohydrolase | | | |
| 884655 | 69668 | Glycyl-tRNA synthetase | GARS | AA629909 | 1.8555 |
| 1759582 | 71366 | Tumor necrosis factor receptor superfamily, member 12A | TNFRSF12A | AI221536 | 1.843 |
| 80549 | 66169 | Pre-B-cell leukemia transcription factor 2 | PBX2 | T59641 | 1.8248 |
| 796152 | 55107 | FERM domain containing 6 | FRMD6 | AA461078 | 1.817 |
| 486787 | 37730 | Calponin 3, acidic | CNN3 | AA043228 | 1.8102 |
| 486035 | 66322 | UDP-N-acteylglucosamine pyrophosphorylase 1 | UAP1 | AA040861 | 1.81 |
| 453722 | 48146 | Insulin-like growth factor 2 mRNA binding protein 2 | IGF2BP2 | AA776408 | 1.8074 |
| 826194 | 57429 | Synaptotagmin-like 2 | SYTL2 | AA521439 | 1.8032 |
| 153614 | 36478 | Interferon-related developmental regulator 1 | IFRD1 | R48587 | 1.7898 |
| 246722 | 66332 | Serpin peptidase inhibitor, clade E (nexin, plasminogen activator inhibitor | SERPINE2 | N59721 | 1.78325 |
| | | type 1), member 2 | | | |
| 825461 | 38120 | Growth arrest and DNA-damage-inducible, beta | GADD45B | AA504354 | 1.76675 |
| 814353 | 75013 | Phorbol-12-myristate-13-acetate-induced protein 1 | PMAIP1 | AA458838 | 1.7318 |
| 858153 | 62027 | Nuclear factor, interleukin 3 regulated | NFIL3 | AA633811 | 1.7284 |
| 223176 | 29985 | MAX dimerization protein 1 | MXD1 | H86558 | 1.726 |
| 2562955 | 49726 | Activating transcription factor 4 (tax-responsive enhancer element B67) | ATF4 | AI986462 | 1.726 |
| 208718 | 67346 | Annexin A1 | ANXA1 | H63077 | 1.7258 |
| 811028 | 30891 | Transmembrane protein 49 | TMEM49 | AA485373 | 1.7085 |
| 768370 | 66511 | Forkhead box C1 | FOXC1 | AA495846 | 1.6732 |
| 712604 | 63037 | | | AA281932 | 1.649 |
| 1466237 | 57168 | Testis derived transcript (3 LIM domains) | TES | AA897151 | 1.6442 |
| 884462 | 47709 | Down syndrome critical region gene 1 | DSCR1 | AA629707 | 1.64325 |
| 298268 | 62906 | B-cell translocation gene 1, anti-proliferative | BTG1 | N70463 | 1.6125 |
| 810441 | 59802 | Fer-1-like 3, myoferlin (C. elegans) | FER1L3 | AA457121 | 1.5912 |
| 855422 | 37702 | Deleted in liver cancer 1 | DLC1 | AA664020 | 1.57625 |
| 813698 | 48210 | Sprouty homolog 2 (Drosophila) | SPRY2 | AA453759 | 1.57125 |
| 2545705 | 46049 | Calponin 3, acidic | CNN3 | AI969128 | 1.559 |
| 810724 | 46995 | Immediate early response 3 | IER3 | AA480815 | 1.551 |
| 502739 | 63866 | 1-acylglycerol-3-phosphate O-acyltransferase 5 (lysophosphatidic acid | AGPAT5 | AA128214 | 1.5404 |
| | | acyltransferase, epsilon) | | | |
| 753610 | 71421 | Translocase of outer mitochondrial membrane 40 homolog (yeast) | TOMM40 | AA478589 | 1.53125 |
| 309864 | 68264 | Jun B proto-oncogene | JUNB | N94468 | 1.5138 |
| 221778 | 68745 | Cysteine conjugate-beta lyase; cytoplasmic (glutamine transaminase K, | CCBL1 | H92216 | 1.5136 |
| | | kyneurenine aminotransferase) | | | |
| 773278 | 63203 | DnaJ (Hsp40) homolog, subfamily B, member 9 | DNAJB9 | AA425320 | 1.51325 |
| 343320 | 44998 | Platelet-derived growth factor beta polypeptide (simian sarcoma viral (v-sis) | PDGFB | W68169 | 1.511 |
| | | oncogene homolog) | | | |
| 45801 | 56270 | V-abl Abelson murine leukemia viral oncogene homolog 2 (arg, Abelson- | ABL2 | H09332 | 1.50425 |
| | | related gene) | | | |
| 280527 | 47295 | Calmodulin binding transcription activator 2 | CAMTA2 | N51651 | 1.4965 |
| 1558707 | 58216 | Tribbles homolog 1 (Drosophila) | TRIB1 | AA977019 | 1.493 |
| 378488 | 66914 | Cysteine-rich, angiogenic inducer, 61 | CYR61 | AA777187 | 1.49125 |
| 744914 | 52777 | MAX dimerization protein 1 | MXD1 | AA625793 | 1.47875 |
| 186768 | 59290 | Chromosome 2 open reading frame 18 | C2orf18 | H51318 | 1.4752 |
| 1621820 | 33905 | Calmodulin binding transcription activator 2 | CAMTA2 | AI003889 | 1.46425 |
| 812965 | 51210 | V-myc myelocytomatosis viral oncogene homolog (avian) | MYC | AA464600 | 1.4564 |
| 1638852 | 45957 | BTB and CNC homology 1, basic leucine zipper transcription factor 1 | BACH1 | AI016618 | 1.44975 |
| 448190 | 41922 | Baculoviral IAP repeat-containing 2 | BIRC2 | AA702174 | 1.4416 |
| 745296 | 35854 | Similar to RPE-spondin | HSUP1 | AA625574 | 1.4385 |
| 949971 | 69549 | Activating transcription factor 4 (tax-responsive enhancer element B67) | ATF4 | AA600217 | 1.4378 |
| 562712 | 64883 | FLJ44896 protein | FLJ44896 | AA086397 | 1.43375 |
| 827168 | 55436 | ATP-binding cassette, sub-family A (ABC1), member 1 | ABCA1 | AA521292 | 1.4316 |
| 122428 | 46082 | Hook homolog 2 (Drosophila) | HOOK2 | T99236 | 1.4306 |
| 2508563 | 68701 | Tumor necrosis factor, alpha-induced protein 3 | TNFAIP3 | AI963014 | 1.42675 |
| 769857 | 61348 | Cystathionine-beta-synthase | CBS | AA430367 | 1.42 |
| 281053 | 64641 | Chromosome 2 open reading frame 18 | C2orf18 | N50907 | 1.4132 |
| 502664 | 51743 | Ras-induced senescence 1 | RIS1 | AA127069 | 1.41025 |
| 342378 | 59466 | Dual specificity phosphatase 5 | DUSP5 | W65461 | 1.40325 |
| 161992 | 32794 | Fer-1-like 3, myoferlin (C. elegans) | FER1L3 | H26176 | 1.38475 |
| 112636 | 49882 | Interleukin 1 receptor accessory protein | IL1RAP | T91161 | 1.38425 |
| 46286 | 32665 | Jumonji domain containing 1C | JMJD1C | H09113 | 1.3828 |
| 489729 | 53714 | V-ets erythroblastosis virus E26 oncogene homolog 1 (avian) | ETS1 | AA101971 | 1.3812 |
| 810567 | 71295 | Rho/rac guanine nucleotide exchange factor (GEF) 2 | ARHGEF2 | AA464578 | 1.3768 |
| 1586016 | 36512 | Death-associated protein kinase 3 | DAPK3 | AA973730 | 1.37475 |
| 34852 | 66645 | Baculoviral IAP repeat-containing 2 | BIRC2 | R19628 | 1.37075 |
| 33611 | 34562 | Ectonucleoside triphosphate diphosphohydrolase 7 | ENTPD7 | R44077 | 1.36625 |
| 148740 | 45432 | Triple functional domain (PTPRF interacting) | TRIO | H12769 | 1.3385 |
| 2562051 | 37291 | G protein-coupled receptor, family C, group 5, member A | GPRC5A | AI984082 | 1.326 |
| 417226 | 47474 | V-myc myelocytomatosis viral oncogene homolog (avian) | MYC | W87741 | 1.32425 |
| 743774 | 57108 | Insulin-like growth factor 2 mRNA binding protein 2 | IGF2BP2 | AA634300 | 1.3135 |
| 360403 | 33612 | Arrestin domain containing 3 | ARRDC3 | AA015658 | 1.3024 |
| 742041 | 44783 | Caldesmon 1 | CALD1 | AA402898 | 1.3016 |
| 149934 | 53247 | Serine/threonine kinase 17a (apoptosis-inducing) | STK17A | H01164 | 1.3008 |
| 1607482 | 52682 | CCAAT/enhancer binding protein (C/EBP), gamma | CEBPG | AI014468 | 1.29875 |
| 770670 | 59369 | Tumor necrosis factor, alpha-induced protein 3 | TNFAIP3 | AA476272 | 1.28575 |
| 280985 | 45649 | Cytoplasmic polyadenylation element binding protein 4 | CPEB4 | N47682 | 1.2845 |
| 240694 | 41682 | UDP-N-acteylglucosamine pyrophosphorylase 1 | UAP1 | H78134 | 1.26625 |
| 143426 | 41590 | Ras homolog gene family, member B | RHOB | R74467 | 1.2578 |
| 122728 | 66067 | Glioblastoma amplified sequence | GBAS | T99032 | 1.25475 |
| 1606557 | 30409 | Four and a half LIM domains 2 | FHL2 | AA995282 | 1.2482 |
| 703739 | 74293 | Nuclear cap binding protein subunit 1, 80 kDa | NCBP1 | AA278749 | 1.237 |
| 813689 | 46577 | Serine/threonine kinase 17a (apoptosis-inducing) | STK17A | AA453754 | 1.2305 |
| 1589468 | 45328 | Epithelial membrane protein 1 | EMP1 | AA975768 | 1.23 |
| 2322367 | 56195 | Reticulon 4 | RTN4 | AI682462 | 1.22225 |
| 2109169 | 55898 | Transmembrane 4 L six family member 19 | TM4SF19 | AI380016 | 1.2186 |
| 815047 | 44109 | Cyclin L1 | CCNL1 | AA465166 | 1.2076 |
| 1502186 | 57596 | LOC440064 | | AA894755 | 1.1982 |
| 731292 | 63877 | Kruppel-like factor 6 | KLF6 | AA416628 | 1.19675 |
| 767994 | 69160 | Striatin, calmodulin binding protein 3 | STRN3 | AA418918 | 1.16525 |
| 741885 | 71049 | Transcription factor binding to IGHM enhancer 3 | TFE3 | AA403035 | 1.1555 |
| 204148 | 53531 | Ribosomal protein S6 kinase, 90 kDa, polypeptide 3 | RPS6KA3 | H55921 | 1.15375 |
| 626716 | 55500 | Elongation factor, RNA polymerase II, 2 | ELL2 | AA191548 | 1.14825 |
| 897159 | 68337 | Coiled-coil domain containing 94 | CCDC94 | AA676962 | 1.1475 |
| 343646 | 44378 | MORN repeat containing 1 | MORN1 | W69471 | 1.1464 |
| 188232 | 34350 | Kruppel-like factor 4 (gut) | KLF4 | H45711 | 1.1454 |
| 34778 | 64674 | Vascular endothelial growth factor | VEGF | R19956 | 1.12575 |
| 163174 | 74052 | Transcription elongation factor A (SII), 1 | TCEA1 | H27379 | 1.1235 |
| 1292136 | 72773 | SEC24 related gene family, member D (S. cerevisiae) | SEC24D | AA705793 | 1.123 |
| 489677 | 43571 | Uridine phosphorylase 1 | UPP1 | AA099568 | 1.11625 |
| 415058 | 69944 | B-cell CLL/lymphoma 10 | BCL10 | W93117 | 1.1148 |
| 782446 | 72189 | Chromosome 21 open reading frame 56 | C21orf56 | AA431571 | 1.1068 |
| 344108 | 41713 | Hypothetical protein LOC148189 | LOC148189 | W73781 | 1.10275 |
| 742672 | 52267 | Polyhomeotic-like 2 (Drosophila) | PHC2 | AA401370 | 1.1026 |
| 377270 | 47590 | Ras homolog gene family, member B | RHOB | AA054975 | 1.0988 |
| 755578 | 47695 | Solute carrier family 7 (cationic amino acid transporter, y+ system), member 5 | SLC7A5 | AA419177 | 1.0915 |
| 1839020 | 30645 | Homo sapiens transcribed sequence | | AI220561 | 1.0892 |
| 754538 | 31707 | DR1-associated protein 1 (negative cofactor 2 alpha) | DRAP1 | AA406285 | 1.084 |
| 841666 | 60481 | Nuclear factor of kappa light polypeptide gene enhancer in B-cells inhibitor, | NFKBIZ | AA487561 | 1.0735 |
| | | zeta | | | |
| 266015 | 66362 | Isoleucine-tRNA synthetase | IARS | N28837 | 1.0735 |
| 1562645 | 76166 | Nuclear factor of kappa light polypeptide gene enhancer in B-cells 2 | NFKB2 | AA952897 | 1.0732 |
| | | (p49/p100) | | | |
| 2016024 | 51892 | ectodysplasin A2 isoform receptor | XEDAR | AI363251 | 1.07275 |
| 502199 | 45719 | Ring finger protein 36 | RNF36 | AA133281 | 1.0726 |
| 2577230 | 64079 | Tenascin C (hexabrachion) | TNC | AW075585 | 1.068 |
| 897733 | 51227 | Solute carrier family 38, member 2 | SLC38A2 | AA598996 | 1.067 |
| 257197 | 47529 | Nuclear receptor binding factor 2 | NRBF2 | N30573 | 1.0522 |
| 134712 | 69650 | Solute carrier family 7 (cationic amino acid transporter, y+ system), member 1 | SLC7A1 | 828280 | 1.05075 |
| 796397 | 40683 | Zinc finger protein 697 | ZNF697 | AA459950 | 1.0496 |
| 593223 | 74223 | Transmembrane protein 49 | TMEM49 | AA159669 | 1.0458 |
| 1525461 | 50655 | Receptor-interacting serine-threonine kinase 2 | RIPK2 | AA913804 | 1.04375 |
| 1606315 | 67812 | Leukocyte-associated immunoglobulin-like receptor 1 | LAIR1 | AA991196 | 1.0402 |
| 431214 | 35871 | Interferon stimulated exonuclease gene 20 kDa-like 1 | ISG20L1 | AA682514 | 1.039 |
| 46463 | 55586 | PRO0149 protein | PRO0149 | H09942 | 1.0364 |
| 1455242 | 42536 | Kruppel-like factor 6 | KLF6 | AA865224 | 1.0326 |
| 796646 | 38993 | Ornithine decarboxylase 1 | ODC1 | AA460115 | 1.0324 |
| 2125485 | 65591 | AHNAK nucleoprotein (desmoyokin) | AHNAK | AI468738 | 1.02075 |
| 358267 | 75317 | Prefoldin subunit 2 | PFDN2 | W95750 | 1.0132 |
| 1950530 | 74834 | Transmembrane protein 107 | TMEM107 | AI338147 | 1.00875 |
| 668442 | 47205 | Discoidin domain receptor family, member 2 | DDR2 | AA243828 | 1.00725 |
| 595037 | 37478 | G protein-coupled receptor, family C, group 5, member A | GPRC5A | AA172400 | 1.00575 |
| 594500 | 50619 | Zinc finger protein 562 | ZNF562 | AA164750 | 1.0032 |
| 279152 | 30039 | PRO0149 protein | PRO0149 | N46831 | 1.0012 |
| 2491434 | 39226 | Solute carrier family 1 (neutral amino acid transporter), member 5 | SLC1A5 | AI973241 | 0.99825 |
| 262695 | 38259 | PALM2-AKAP2 protein | PALM2- | H99415 | 0.99475 |
| | | | AKAP2 | | |
| 344191 | 53176 | PRO0149 protein | PRO0149 | W69799 | 0.9895 |
| 2013515 | 57896 | Serum/glucocorticoid regulated kinase | SGK | AI375353 | 0.9885 |
| 2495781 | 30418 | Tissue factor pathway inhibitor (lipoprotein-associated coagulation | TFPI | AI985214 | 0.98325 |
| | | inhibitor) | | | |
| 120533 | 64560 | Homeodomain interacting protein kinase 2 | HIPK2 | T95411 | 0.98 |
| 771220 | 30478 | V-rel reticuloendotheliosis viral oncogene homolog A, nuclear factor of | RELA | AA443546 | 0.9744 |
| | | kappa light polypeptide gene enhancer in B-cells 3, p65 (avian) | | | |
| 809533 | 42207 | Hypothetical protein MGC14376 | MGC14376 | AA454584 | 0.9738 |
| 2211651 | 71932 | Zinc finger protein 217 | ZNF217 | AI559473 | 0.973 |
| 247559 | 65985 | Chromosome 14 open reading frame 43 | C14orf43 | N54188 | 0.9695 |
| 795395 | 51940 | Josephin domain containing 3 | JOSD3 | AA453287 | 0.9675 |
| 773220 | 34634 | O-linked N-acetylglucosamine (GlcNAc) transferase (UDP-N- | OGT | AA425655 | 0.96325 |
| | | acetylglucosamine:polypeptide-N-acetylglucosaminyl transferase) | | | |
| 768454 | 41896 | Cytoplasmic polyadenylation element binding protein 4 | CPEB4 | AA495924 | 0.9628 |
| 796309 | 65950 | Similar to RPE-spondin | HSUP1 | AA461309 | 0.9616 |
| 858761 | 49846 | CDNA FLJ30740 fis, clone FEBRA2000319 | | AA779048 | 0.95575 |
| 796090 | 42344 | Mannosidase, alpha, class 2C, member 1 | MAN2C1 | AA460369 | 0.9485 |
| 85202 | 69655 | Serine dehydratase | SDS | T71363 | 0.9464 |
| 324510 | 63815 | Restin-like 2 | RSNL2 | AA284277 | 0.9445 |
| 840567 | 69418 | Transmembrane 4 L six family member 1 | TM4SF1 | AA487893 | 0.9435 |
| 855557 | 49488 | Protein kinase (cAMP-dependent, catalytic) inhibitor gamma | PKIG | AA664210 | 0.942 |
| 1607039 | 41876 | Eukaryotic translation initiation factor 1 | EIF1 | AA988313 | 0.938 |
| 1457276 | 63481 | G protein-coupled receptor, family C, group 5, member A | GPRC5A | AA911832 | 0.938 |
| 85194 | 69009 | TIGA1 | TIGA1 | | 0.9338 |
| 339235 | 37193 | TSC22 domain family, member 2 | TSC22D2 | W60983 | 0.9335 |
| 53391 | 43165 | Hypothetical protein LOC148189 | LOC148189 | R16241 | 0.932 |
| 344672 | 39695 | Zinc finger, CCHC domain containing 9 | ZCCHC9 | W74565 | 0.9302 |
| 186757 | 58642 | Discoidin domain receptor family, member 2 | DDR2 | H51317 | 0.92875 |
| 1896729 | 68440 | Fibronectin type III domain containing 3B | FNDC3B | AI298110 | 0.9284 |
| 856878 | 68299 | Chromosome 20 open reading frame 121 | C20orf121 | AA669593 | 0.9262 |
| 2541203 | 30964 | Solute carrier family 7 (cationic amino acid transporter, y+ system), member 5 | SLC7A5 | AW028368 | 0.9256 |
| 41174 | 36619 | Hypothetical protein FLJ21657 | FLJ21657 | R56106 | 0.92525 |
| 124893 | 48917 | Insulin-like growth factor 2 mRNA binding protein 2 | IGF2BP2 | R06121 | 0.92475 |
| AB007899 | 80988 | Neural precursor cell expressed, developmentally down-regulated 4-like | NEDD4L | AB007899 | 0.9198 |
| 506648 | 60412 | Cyclin-dependent kinase-like 5 | CDKL5 | AA708794 | 0.9134 |
| 1702742 | 54269 | Homo sapiens transcribed sequence | | AI096953 | 0.9116 |
| 182177 | 63745 | ADAM metallopeptidase domain 17 (tumor necrosis factor, alpha, | ADAM17 | H28287 | 0.9075 |
| | | converting enzyme) | | | |
| 233645 | 65451 | ESTs, Weakly similar to ubiquitously transcribed tetratricopeptide repeat gene, Y | | H79007 | 0.907 |
| | | chromosome; Ubiquitously transcribed TPR gene on Y chromosome [Homo sapiens] | | | |
| 854593 | 50154 | KIAA0310 | KIAA0310 | AA669152 | 0.9032 |
| 328868 | 34907 | CD44 molecule (Indian blood group) | CD44 | W45275 | 0.894 |
| 361668 | 34247 | Metastasis associated lung adenocarcinoma transcript 1 (non-coding RNA) | MALAT1 | W96187 | 0.892 |
| 417867 | 55139 | X-box binding protein 1 | XBP1 | W90128 | 0.8895 |
| 44477 | 38949 | Vascular cell adhesion molecule 1 | VCAM1 | H07071 | 0.886 |
| 1670291 | 34888 | Oxysterol binding protein-like 6 | OSBPL6 | AI094626 | 0.88425 |
| 262772 | 39566 | Abl-interactor 1 | ABI1 | H99626 | 0.86975 |
| 249070 | 59700 | Methylenetetrahydrofolate dehydrogenase (NADP+ dependent) 1-like | MTHFD1L | H80063 | 0.867 |
| 841415 | 66424 | LIM domain and actin binding 1 | LIMA1 | AA487557 | 0.86375 |
| 2013178 | 55599 | Death inducer-obliterator 1 | DIDO1 | AI359211 | 0.86275 |
| 470114 | 47986 | Mediator of RNA polymerase II transcription, subunit 8 homolog (S. cerevisiae) | MED8 | AA029295 | 0.86175 |
| 23185 | 30852 | Tenascin C (hexabrachion) | TNC | T77595 | 0.8604 |
| 784168 | 53759 | PDZ and LIM domain 5 | PDLIM5 | AA432103 | 0.8586 |
| 951303 | 76018 | Protein kinase, AMP-activated, beta 2 non-catalytic subunit | PRKAB2 | AA620527 | 0.8574 |
| 296030 | 70389 | Tripartite motif-containing 25 | TRIM25 | N73575 | 0.85125 |
| 343744 | 55843 | H1 histone family, member 0 | H1F0 | W69399 | 0.85075 |
| 810411 | 58817 | Zinc finger, AN1-type domain 3 | ZFAND3 | AA457102 | 0.85075 |
| 162077 | 41628 | Pleckstrin homology-like domain, family A, member 1 | PHLDA1 | H26271 | 0.84925 |
| 263200 | 34942 | Discoidin, CUB and LCCL domain containing 2 | DCBLD2 | H99544 | 0.8478 |
| 549349 | 32461 | Solute carrier family 38, member 2 | SLC38A2 | AA081106 | 0.8446 |
| 273048 | 71230 | Muscleblind-like 2 (Drosophila) | MBNL2 | N36402 | 0.844 |
| 1627705 | 32777 | STAM binding protein-like 1 | STAMBPL1 | AI017607 | 0.8434 |
| 841703 | 71611 | Transferrin receptor (p90, CD71) | TFRC | AA488721 | 0.8385 |
| 45641 | 45451 | Mitogen-activated protein kinase kinase 3 | MAP2K3 | H08749 | 0.8366 |
| 2042739 | 33038 | Serpin peptidase inhibitor, clade A (alpha-1 antiproteinase, antitrypsin), | SERPINA1 | AI375788 | 0.83375 |
| | | member 1 | | | |
| 120892 | 50755 | Hypothetical protein FLJ43663 | FLJ43663 | T95898 | 0.8275 |
| 854079 | 65288 | Actinin, alpha 1 | ACTN1 | AA669042 | 0.82375 |
| 2109639 | 65904 | Brevican | BCAN | AI392746 | 0.8184 |
| 502396 | 29956 | Kruppel-like factor 6 | KLF6 | AA156946 | 0.81325 |
| 810802 | 30913 | Restin (Reed-Steinberg cell-expressed intermediate filament-associated | RSN | AA458868 | 0.8025 |
| | | protein) | | | |
| 782853 | 67226 | Itchy homolog E3 ubiquitin protein ligase (mouse) | ITCH | AA448286 | 0.80125 |
| 209224 | 63354 | Dual-specificity tyrosine-(Y)-phosphorylation regulated kinase 3 | DYRK3 | H62028 | 0.80075 |
| 837953 | 30174 | Neural precursor cell expressed, developmentally down-regulated 4-like | NEDD4L | AA458578 | 0.78975 |
| 130242 | 43187 | Cyclin-dependent kinase 7 (MO15 homolog, Xenopus laevis, cdk-activating | CDK7 | R22625 | 0.7894 |
| | | kinase) | | | |
| 703916 | 48026 | B-cell CLL/lymphoma 10 | BCL10 | AA279060 | 0.77925 |
| 1629227 | 50070 | Gap junction protein, alpha 7, 45 kDa (connexin 45) | GJA7 | AI003367 | 0.775 |
| 789376 | 41485 | Thioredoxin reductase 1 | TXNRD1 | AA453335 | 0.767 |
| 841620 | 49658 | Dihydropyrimidinase-like 2 | DPYSL2 | AA487674 | 0.76625 |
| 745402 | 67964 | Casein kinase 1, alpha 1 | CSNK1A1 | AA625758 | 0.7652 |
| 67070 | 34241 | Step II splicing factor SLU7 | SLU7 | T70429 | 0.7635 |
| 212198 | 33469 | Tumor protein p53 binding protein, 2 | TP53BP2 | H69153 | 0.763 |
| 626068 | 61775 | Exportin, tRNA (nuclear export receptor for tRNAs) | XPOT | AA211459 | 0.7628 |
| 267460 | 39760 | Golgi transport 1 homolog B (S. cerevisiae) | GOLT1B | N25241 | 0.761 |
| 208699 | 67014 | KIAA1949 | KIAA1949 | H61003 | 0.76075 |
| 2105465 | 52407 | Signal sequence receptor, gamma (translocon-associated protein gamma) | SSR3 | AI394435 | 0.7585 |
| 824052 | 59749 | Chromosome 6 open reading frame 1 | C6orf1 | AA491208 | 0.75 |
| 1842793 | 37318 | CD44 molecule (Indian blood group) | CD44 | AI221846 | 0.7482 |
| 730362 | 48915 | PRO0149 protein | PRO0149 | AA469950 | 0.748 |
| 32393 | 65144 | Vacuolar protein sorting 37 homolog B (S. cerevisiae) | VPS37B | R43481 | 0.74675 |
| 489594 | 53356 | MORC family CW-type zinc finger 4 | MORC4 | AA099523 | 0.73725 |
| 824602 | 34456 | Pyrin and HIN domain family, member 1 | PYHIN1 | AA491191 | 0.7298 |
| 1914168 | 33706 | Hypothetical protein FLJ20558 | FLJ20558 | AI309257 | 0.7234 |
| 1636606 | 51556 | UDP glucuronosyltransferase 2 family, polypeptide B7 | UGT2B7 | AI000188 | 0.721 |
| 382773 | 36000 | Metastasis associated lung adenocarcinoma transcript 1 (non-coding RNA) | MALAT1 | AA064973 | 0.7146 |
| 436191 | 70356 | Hypothetical protein KIAA1434 | RP5- | AA703277 | 0.71425 |
| | | | 1022P6.2 | | |
| 322561 | 33051 | Ribosomal protein L31 | RPL31 | W15277 | 0.698 |
| 135640 | 40724 | Syntaxin 3 | STX3 | R32377 | 0.696 |
| 744647 | 41309 | Catenin (cadherin-associated protein), alpha-like 1 | CTNNAL1 | AA621315 | 0.6952 |
| 489623 | 54672 | Early B-cell factor | EBF | | 0.6952 |
| 32687 | 72300 | Brevican | BCAN | R43547 | 0.6878 |
| 1915149 | 44537 | EST | | AI371376 | 0.68575 |
| 796760 | 70445 | glutamine-fructose-6-phosphate transaminase 1 | GFPT1 | AA460722 | 0.677 |
| 1607765 | 53990 | BCL2/adenovirus E1B 19 kDa interacting protein 1 | BNIP1 | AA989473 | 0.675 |
| 129644 | 72439 | Abl-interactor 1 | ABI1 | R16667 | 0.672 |
| 1642189 | 51968 | Oncostatin M receptor | OSMR | AI018655 | 0.67025 |
| 826253 | 30520 | RAB3 GTPase activating protein subunit 2 (non-catalytic) | RAB3GAP2 | AA520985 | 0.6658 |
| 246035 | 50320 | Ankyrin repeat domain 38 | ANKRD38 | N55540 | 0.6576 |
| 1636108 | 42443 | Phosphoserine aminotransferase 1 | PSAT1 | AI015679 | 0.6558 |
| 112571 | 69527 | Exostoses (multiple) 1 | EXT1 | T91083 | 0.65575 |
| 1632015 | 34625 | Zinc finger protein 300 | ZNF300 | AA994690 | 0.6555 |
| 755821 | 38565 | Nuclear factor (erythroid-derived 2)-like 1 | NFE2L1 | AA496576 | 0.65225 |
| 1486013 | 54916 | Isoleucine-tRNA synthetase | IARS | AA912034 | 0.6502 |
| 755581 | 48017 | Eukaryotic translation initiation factor 2-alpha kinase 1 | EIF2AK1 | AA419143 | 0.645 |
| 1603346 | 47502 | Erythrocyte membrane protein band 4.1 (elliptocytosis 1, RH-linked) | EPB41 | AA987359 | 0.642 |
| 510790 | 59081 | Tyrosyl-tRNA synthetase | YARS | AA102053 | 0.6392 |
| 2557762 | 38427 | Pyrroline-5-carboxylate reductase 1 | PYCR1 | AW050510 | 0.63075 |
| 1857589 | 71327 | | | AI269390 | 0.6286 |
| 361048 | 67683 | Staphylococcal nuclease domain containing 1 | SND1 | AA017382 | 0.62575 |
| 877776 | 43874 | Glutamic-oxaloacetic transaminase 1, soluble (aspartate aminotransferase 1) | GOT1 | AA626786 | 0.625 |
| 505944 | 54228 | Zinc finger, DHHC-type containing 21 | ZDHHC21 | AA778351 | 0.6245 |
| 1702847 | 54929 | Homo sapiens transcribed sequences | | AI147705 | 0.624 |
| 843008 | 48982 | Eukaryotic translation initiation factor 1B | EIF1B | AA488391 | 0.6208 |
| 40303 | 29762 | SHC (Src homology 2 domain containing) transforming protein 1 | SHC1 | R52961 | 0.616 |
| 773290 | 72706 | Neurofilament, heavy polypeptide 200 kDa | NEFH | AA425336 | 0.60975 |
| 743804 | 58420 | Sec23 homolog B (S. cerevisiae) | SEC23B | AA634360 | 0.6085 |
| 2013881 | 69040 | Triple functional domain (PTPRF interacting) | TRIO | AI359699 | 0.6036 |
| 50506 | 73098 | Mitogen-activated protein kinase 6 | MAPK6 | H17504 | 0.5772 |
| 1586340 | 56165 | NECAP endocytosis associated 1 | NECAP1 | AA974348 | 0.571 |
| 837892 | 66913 | Thyroid hormone receptor associated protein 1 | THRAP1 | AA434084 | 0.5665 |
| 745175 | 61932 | Spermatid perinuclear RNA binding protein | STRBP | AA626730 | 0.556 |
| 82879 | 75312 | Mannose-binding lectin (protein C) 2, soluble (opsonic defect) | MBL2 | T69359 | 0.555 |
| 299442 | 35301 | Chromosome 8 open reading frame 41 | C8orf41 | W05442 | 0.5466 |
| 34849 | 66316 | Eukaryotic translation elongation factor 2 | EEF2 | R20379 | 0.5418 |
| 175950 | 33655 | 3-phosphoinositide dependent protein kinase-1 | PDPK1 | H40880 | 0.5386 |
| 1553560 | 48506 | Hypothetical protein FLJ36031 | FLJ36031 | AA962436 | 0.532 |
| 178805 | 32984 | Sulfiredoxin 1 homolog (S. cerevisiae) | SRXN1 | H49601 | 0.5316 |
| 251732 | 54537 | Hypothetical protein LOC285550 | LOC285550 | H96902 | 0.526 |
| 2509911 | 70989 | Synaptogyrin 2 | SYNGR2 | AI961866 | 0.5176 |
| 366067 | 55073 | Cerebellar degeneration-related protein 2, 62 kDa | CDR2 | AA074613 | 0.51575 |
| 366484 | 40941 | GTP binding protein 1 | GTPBP1 | AA026413 | 0.5058 |
| 1636707 | 60737 | Eukaryotic translation initiation factor 3, subunit 3 gamma, 40 kDa | EIF3S3 | AI017703 | 0.5006 |

Table 2B

| 2028984 | 62265 | Chemokine (C—X—C motif) ligand 12 (stromal cell-derived factor 1) | CXCL12 | AI263201 | −0.50525 |
|---|---|---|---|---|---|
| 50339 | 72417 | Hypothetical protein LOC550643 | LOC550643 | H16780 | −0.50775 |
| 1049282 | 58147 | BTB (POZ) domain containing 14A | BTBD14A | AA620746 | −0.5104 |
| 815183 | 36936 | Hippocampus abundant transcript-like 1 | HIATL1 | AA481152 | −0.52175 |
| 108265 | 44730 | Transmembrane protein 99 | TMEM99 | T70541 | −0.5232 |
| 789369 | 41158 | Inhibitor of DNA binding 4, dominant negative helix-loop-helix protein | ID4 | AA464856 | −0.5292 |
| 360547 | 36231 | Hydroxysteroid dehydrogenase like 1 | HSDL1 | AA015978 | −0.5365 |
| 773375 | 49391 | DKFZP564J0863 protein | DKFZP564J0863 | AA425723 | −0.5368 |
| 2018332 | 41471 | Protein kinase, cAMP-dependent, regulatory, type I, alpha (tissue specific | PRKAR1A | AI362806 | −0.5392 |
| | | extinguisher 1) | | | |
| 1505534 | 43141 | Nephroblastoma overexpressed gene | NOV | AA910443 | −0.54075 |
| 771295 | 42919 | Ubiquitin-conjugating enzyme E2G 2 (UBC7 homolog, yeast) | UBE2G2 | AA443634 | −0.54775 |
| 1947289 | 54553 | Homo sapiens transcribed sequence | | AI350851 | −0.548 |
| 898237 | 57063 | HLA-B associated transcript 3 | BAT3 | AA598629 | −0.5502 |
| 266361 | 68038 | Melan-A | MLANA | N26562 | −0.551 |
| 200402 | 40542 | Family with sequence similarity 83, member D | FAM83D | R96941 | −0.5566 |
| 178922 | 42477 | Chromosome 9 open reading frame 58 | C9orf58 | H48148 | −0.5662 |
| 505289 | 42409 | Angiotensin II receptor-associated protein | AGTRAP | AA152101 | −0.574 |
| 200418 | 41189 | Intraflagellar transport 122 homolog (Chlamydomonas) | IFT122 | R97234 | −0.585 |
| 810063 | 65799 | Growth factor, augmenter of liver regeneration (ERV1 homolog, S. cerevisiae) | GFER | AA465021 | −0.58725 |
| 2544350 | 33608 | Catenin (cadherin-associated protein), beta 1, 88 kDa | CTNNB1 | AW058504 | −0.59 |
| 811024 | 30238 | Bone marrow stromal cell antigen 2 | BST2 | AA485371 | −0.5935 |
| 306743 | 70261 | Twisted gastrulation homolog 1 (Drosophila) | TWSG1 | N91767 | −0.6 |
| 42831 | 54079 | Latent transforming growth factor beta binding protein 3 | LTBP3 | R60197 | −0.6094 |
| 884540 | 59177 | Sorting nexin 12 | SNX12 | AA629796 | −0.611 |
| 234191 | 73256 | Son of sevenless homolog 1 (Drosophila) | SOS1 | H64324 | −0.61375 |
| 951241 | 73722 | Nucleolar and spindle associated protein 1 | NUSAP1 | AA620485 | −0.617 |
| 213211 | 41603 | Chloride channel 3 | CLCN3 | H69811 | −0.62075 |
| 53158 | 66810 | 6-phosphofructo-2-kinase/fructose-2,6-biphosphatase 2 | PFKFB2 | R16146 | −0.6214 |
| 2018154 | 31334 | Electron-transfer-flavoprotein, beta polypeptide | ETFB | AI364521 | −0.6326 |
| 362181 | 65691 | Zinc finger protein 291 | ZNF291 | AA001504 | −0.6362 |
| 825411 | 35176 | Heterogeneous nuclear ribonucleoprotein M | HNRPM | AA504272 | −0.65125 |
| 788609 | 30326 | Chromosome 7 open reading frame 38 | C7orf38 | AA452899 | −0.65575 |
| 487373 | 41357 | ATP synthase, H+ transporting, mitochondrial F0 complex, subunit C1 | ATP5G1 | AA046701 | −0.6558 |
| | | (subunit 9) | | | |
| 1457251 | 62820 | Dermatan 4 sulfotransferase 1 | D4ST1 | AA922082 | −0.65675 |
| 471655 | 52859 | Chromosome 14 open reading frame 24 | C14orf24 | AA035549 | −0.6602 |
| 79045 | 58007 | Solute carrier family 44, member 1 | SLC44A1 | T61913 | −0.6626 |
| 147630 | 33941 | Chromodomain helicase DNA binding protein 9 | CHD9 | R81880 | −0.6642 |
| 345103 | 52828 | EPH receptor B2 | EPHB2 | W72792 | −0.66625 |
| 41463 | 35907 | Solute carrier family 44, member 1 | SLC44A1 | R59173 | −0.6684 |
| 126474 | 32167 | Mevalonate kinase (mevalonic aciduria) | MVK | R06716 | −0.673 |
| 51331 | 30354 | Myosin VA (heavy polypeptide 12, myoxin) | MYO5A | H20809 | −0.6754 |
| 248975 | 51177 | Hypothetical protein LOC151162 | LOC151162 | H79970 | −0.6815 |
| 1685029 | 52873 | HBS1-like (S. cerevisiae) | HBS1L | AI003293 | −0.6832 |
| 815556 | 42453 | Neuropilin (NRP) and tolloid (TLL)-like 2 | NETO2 | AA456821 | −0.6854 |
| 251875 | 65673 | Myelin expression factor 2 | MYEF2 | H96671 | −0.6864 |
| 811737 | 37566 | Chromosome 13 open reading frame 8 | C13orf8 | AA463267 | −0.6892 |
| 245386 | 51326 | MRNA full length insert cDNA clone EUROIMAGE 200999 | | N54993 | −0.698 |
| 845604 | 44336 | Vpr (HIV-1) binding protein | VPRBP | AA644335 | −0.702 |
| 2502789 | 71364 | Nephroblastoma overexpressed gene | NOV | AW008840 | −0.7034 |
| 809535 | 42532 | Splicing factor, arginine/serine-rich 2 | SFRS2 | AA454585 | −0.7076 |
| 825470 | 46952 | Topoisomerase (DNA) II alpha 170 kDa | TOP2A | AA504348 | −0.7142 |
| 1837950 | 48384 | hypothetical protein FLJ12973 | FLJ12973 | AI220472 | −0.71475 |
| 1635186 | 68394 | Vacuolar protein sorting 13 homolog D (S. cerevisiae) | VPS13D | AI005042 | −0.71775 |
| 1849998 | 68638 | Cell division cycle associated 2 | CDCA2 | AI248208 | −0.719 |
| 1859075 | 34072 | EST | | AI201652 | −0.7242 |
| 489208 | 66189 | Chromosome 16 open reading frame 34 | C16orf34 | AA045658 | −0.72525 |
| 1857873 | 61681 | Integrin, beta 8 | ITGB8 | AI246160 | −0.7288 |
| 197933 | 60642 | Nucleoporin like 2 | NUPL2 | R96358 | −0.73125 |
| 815225 | 46096 | Heat shock factor binding protein 1 | HSBP1 | AA481263 | −0.735 |
| 1034676 | 66085 | Homo sapiens transcribed sequence with strong similarity to protein pdb: 1BGM (E. coli) O | | AA779865 | −0.735 |
| | | Chain O, Beta-Galactosidase (Chains I-P) | | | |
| 429626 | 31417 | MAX gene associated | MGA | AA011551 | −0.74 |
| 42408 | 44077 | 1-acylglycerol-3-phosphate O-acyltransferase 3 | AGPAT3 | R61733 | −0.743 |
| 502413 | 30935 | Dpy-19-like 2 (C. elegans) | DPY19L2 | AA134696 | −0.75575 |
| 40718 | 39611 | Homo sapiens transcribed sequences | | R55747 | −0.756 |
| 149013 | 58541 | Adenosylmethionine decarboxylase 1 | AMD1 | R82299 | −0.758 |
| 1840753 | 44284 | protocadherin beta 16 | PCDHB16 | AI218958 | −0.7648 |
| 454698 | 49121 | Frizzled homolog 4 (Drosophila) | FZD4 | AA677200 | −0.765 |
| 79761 | 52373 | Thymopoietin | TMPO | T63980 | −0.7846 |
| 160233 | 44296 | Thymopoietin | TMPO | H21943 | −0.7848 |
| 26443 | 30381 | START domain containing 7 | STARD7 | R37351 | −0.7862 |
| 739193 | 73677 | Cellular retinoic acid binding protein 1 | CRABP1 | AA421218 | −0.79225 |
| 823815 | 54221 | Cartilage associated protein | CRTAP | AA490280 | −0.79425 |
| 812238 | 66326 | Hypothetical protein MGC4692 | MGC4692 | AA455039 | −0.79425 |
| 139354 | 31408 | CXXC finger 5 | CXXC5 | R63735 | −0.8002 |
| 809588 | 53334 | Gamma-glutamyl hydrolase (conjugase, folylpolygammaglutamyl | GGH | AA455800 | −0.8048 |
| | | hydrolase) | | | |
| 591116 | 75549 | Nucleoporin like 1 | NUPL1 | AA158352 | −0.8132 |
| 813387 | 38422 | NAD(P)H dehydrogenase, quinone 1 | NQO1 | AA455538 | −0.8166 |
| 84560 | 46386 | Tensin 3 | TNS3 | T74023 | −0.8212 |
| 488956 | 46522 | CUG triplet repeat, RNA binding protein 2 | CUGBP2 | AA047257 | −0.8218 |
| 897727 | 49909 | MAF1 homolog (S. cerevisiae) | MAF1 | AA598994 | −0.8305 |
| 263916 | 73335 | Transcribed sequences | | H99855 | −0.83775 |
| 755975 | 49364 | Dystroglycan 1 (dystrophin-associated glycoprotein 1) | DAG1 | AA496691 | −0.83775 |
| 130931 | 30396 | Mannosidase, alpha, class 1A, member 2 | MAN1A2 | R22905 | −0.83875 |
| 431462 | 46702 | Lectin, galactoside-binding, soluble, 13 (galectin 13) | LGALS13 | AA706870 | −0.84525 |
| 269791 | 34817 | Dopachrome tautomerase (dopachrome delta-isomerase, tyrosine-related | DCT | N27147 | −0.8518 |
| | | protein 2) | | | |
| 2016426 | 51592 | KIAA0664 | KIAA0664 | AI363909 | −0.85375 |
| 768569 | 63514 | Hypothetical protein LOC286334 | LOC286334 | AA425105 | −0.8562 |
| 713347 | 51875 | Leucine-rich repeat kinase 2 | LRRK2 | AA283609 | −0.8566 |
| 2014525 | 52600 | Clone IMAGE: 5311129, mRNA | | AI362218 | −0.8568 |
| 700299 | 51995 | Wiskott-Aldrich syndrome protein interacting protein | WASPIP | AA283699 | −0.8604 |
| 704410 | 35216 | Three prime repair exonuclease 1 | TREX1 | AA279658 | −0.861 |
| 823902 | 76168 | Tumor necrosis factor receptor superfamily, member 21 | TNFRSF21 | AA490494 | −0.8622 |
| 855563 | 57689 | V-erb-b2 erythroblastic leukemia viral oncogene homolog 3 (avian) | ERBB3 | AA664212 | −0.864 |
| 2449395 | 58356 | Aldo-keto reductase family 1, member C2 (dihydrodiol dehydrogenase 2; | AKR1C2 | AI924357 | −0.865 |
| | | bile acid binding protein; 3-alpha hydroxysteroid dehydrogenase, type III) | | | |
| 768939 | 63359 | Teashirt family zinc finger 1 | TSHZ1 | AA426522 | −0.865 |
| 123474 | 66051 | Stearoyl-CoA desaturase (delta-9-desaturase) | SCD | R00707 | −0.87325 |
| 2111914 | 49069 | Hypothetical protein FLJ21901 | FLJ21901 | AI392678 | −0.8782 |
| 884511 | 57220 | Cytochrome c oxidase subunit VIIb | COX7B | AA629999 | −0.87975 |
| 460150 | 66797 | F-box and leucine-rich repeat protein 13 | FBXL13 | AA676859 | −0.88025 |
| 1501546 | 36638 | Pleckstrin homology domain containing, family F (with FYVE domain) | PLEKHF2 | AA886792 | −0.8846 |
| | | member 2 | | | |
| 754273 | 43512 | Sarcoglycan, delta (35 kDa dystrophin-associated glycoprotein) | SGCD | AA479286 | −0.8855 |
| 840878 | 45144 | 24-dehydrocholesterol reductase | DHCR24 | AA482324 | −0.8912 |
| 49941 | 65261 | Ubiquitin specific peptidase 40 | USP40 | H29211 | −0.89325 |
| 131984 | 65802 | TBC1 (tre-2/USP6, BUB2, cdc16) domain family, member 1 | TBC1D1 | R32437 | −0.8948 |
| 767798 | 37376 | ATX1 antioxidant protein 1 homolog (yeast) | ATOX1 | AA418755 | −0.898 |
| 269029 | 57435 | Guanine nucleotide binding protein (G protein), gamma 2 | GNG2 | N26108 | −0.9006 |
| 46827 | 39813 | Vav 3 oncogene | VAV3 | H10045 | −0.9075 |
| 450515 | 73544 | Solute carrier family 26 (sulfate transporter), member 2 | SLC26A2 | AA704222 | −0.9114 |
| 2131507 | 69462 | TBC1 domain family, member 16 | TBC1D16 | AI431804 | −0.9208 |
| 838899 | 64920 | Tripartite motif-containing 2 | TRIM2 | AA464935 | −0.922 |
| 950355 | 32809 | Ovostatin 2 | OVOS2 | AA600184 | −0.9275 |
| 1055146 | 71890 | DEP domain containing 6 | DEPDC6 | AA621364 | −0.93 |
| 452848 | 30492 | Kelch-like ECH-associated protein 1 | KEAP1 | AA704816 | −0.9362 |
| 811069 | 52832 | Establishment of cohesion 1 homolog 2 (S. cerevisiae) | ESCO2 | AA485454 | −0.9392 |
| 950594 | 75710 | SERTA domain containing 4 | SERTAD4 | AA608531 | −0.9485 |
| 1914667 | 45487 | Transmembrane protein 60 | TMEM60 | AI310513 | −0.9492 |
| 415525 | 51822 | Hermansky-Pudlak syndrome 1 | HPS1 | W80375 | −0.9535 |
| 341840 | 35233 | ADAM metallopeptidase with thrombospondin type 1 motif, 14 | ADAMTS14 | W60649 | −0.9575 |
| 489850 | 64853 | Cytochrome P450, family 2, subfamily U, polypeptide 1 | CYP2U1 | AA099864 | −0.9575 |
| 32801 | 63492 | N-acetylglucosamine-1-phosphate transferase, alpha and beta subunits | GNPTAB | R43609 | −0.9606 |
| 1754220 | 40056 | | | AI204285 | −0.96975 |
| 839048 | 74747 | Immunoglobulin superfamily, member 4 | IGSF4 | AA487505 | −0.9755 |
| 746051 | 66628 | Chromosome 5 open reading frame 24 | C5orf24 | AA482026 | −0.97575 |
| 322723 | 61292 | CDNA FLJ34046 fis, clone FCBBF2007610 | | W15465 | −0.9785 |
| 1602927 | 46516 | Hypothetical protein MGC35048 | MGC35048 | AA989072 | −0.985 |
| 156343 | 70201 | Mitogen-activated protein kinase kinase kinase 3 | MAP3K3 | R72632 | −0.9882 |
| 288919 | 65853 | Sestrin 3 | SESN3 | N62640 | −0.99175 |
| 366071 | 55400 | TM2 domain containing 2 | TM2D2 | AA074614 | −1.009 |
| 1759290 | 59576 | Chondroitin beta1,4 N-acetylgalactosaminyltransferase | ChGn | AI219094 | −1.01125 |
| 41432 | 34604 | Hypothetical protein LOC283824 | LOC283824 | R56916 | −1.0168 |
| 1542749 | 44721 | Homo sapiens transcribed sequence | | AA909118 | −1.0176 |
| 289423 | 63833 | Putative homeodomain transcription factor 2 | PHTF2 | N63954 | −1.0374 |
| 754346 | 55629 | Transcribed sequences | | AA436138 | −1.0428 |
| 823655 | 67386 | Chromosome 4 open reading frame 18 | C4orf18 | AA496988 | −1.0442 |
| 713129 | 51843 | Granzyme A (granzyme 1, cytotoxic T-lymphocyte-associated serine | GZMA | AA283007 | −1.04575 |
| | | esterase 3) | | | |
| 234527 | 49946 | Carbamoyl-phosphate synthetase 1, mitochondrial | CPS1 | H77554 | −1.05275 |
| 753184 | 52472 | SRY (sex determining region Y)-box 9 (campomelic dysplasia, autosomal | SOX9 | AA400739 | −1.059 |
| | | sex-reversal) | | | |
| 43733 | 52063 | Glycogenin 2 | GYG2 | H04789 | −1.07625 |
| 785540 | 61608 | Calpain, small subunit 2 | CAPNS2 | AA450334 | −1.10275 |
| 293729 | 33789 | Signal peptide peptidase 3 | UNQ1887 | N63835 | −1.1435 |
| 44975 | 40267 | Isopentenyl-diphosphate delta isomerase 1 | IDI1 | H08899 | −1.1592 |
| 1838959 | 50917 | Homo sapiens transcribed sequence with moderate similarity to protein sp: P12004 | | AI223432 | −1.17225 |
| | | (H. sapiens) PCNA_HUMAN Proliferating cell nuclear antigen (PCNA) (Cyclin) | | | |
| 24948 | 71763 | Calpain, small subunit 2 | CAPNS2 | R38885 | −1.1804 |
| 1901429 | 48957 | Calpain, small subunit 2 | CAPNS2 | AI302534 | −1.2128 |
| 257323 | 73488 | Hypothetical protein from EUROIMAGE 588495 | LOC58489 | N26928 | −1.215 |
| 204737 | 38601 | Snail homolog 2 (Drosophila) | SNAI2 | H57309 | −1.2298 |
| 2094668 | 58728 | Calpain, small subunit 2 | CAPNS2 | AI420081 | −1.2535 |
| 183556 | 47965 | Gap junction protein, alpha 4, 37 kDa (connexin 37) | GJA4 | H44032 | −1.25425 |
| 795612 | 59438 | Serologically defined colon cancer antigen 33 | SDCCAG33 | AA460005 | −1.2725 |
| 731118 | 45582 | Signal-regulatory protein alpha | SIRPA | AA417279 | −1.314 |
| 823912 | 38127 | Ubiquitin-like 3 | UBL3 | AA490497 | −1.31625 |
| 2016775 | 74848 | G protein-coupled receptor, family C, group 5, member B | GPRC5B | AI356028 | −1.3818 |
| 26259 | 55321 | Basic helix-loop-helix domain containing, class B, 9 | BHLHB9 | R20547 | −1.38475 |
| 1558675 | 57886 | SRY (sex determining region Y)-box 10 | SOX10 | AA976578 | −1.39475 |
| 773335 | 39241 | Immunoglobulin superfamily, member 3 | IGSF3 | AA425437 | −1.3966 |
| 842980 | 47678 | Developmentally regulated GTP binding protein 1 | DRG1 | AA488336 | −1.4305 |
| 826301 | 40994 | DnaJ (Hsp40) homolog, subfamily C, member 4 | DNAJC4 | AA521015 | −1.437 |
| 271985 | 63568 | Tyrosinase (oculocutaneous albinism IA) | TYR | N42770 | −1.5315 |
| 291985 | 41074 | Solute carrier family 26 (sulfate transporter), member 2 | SLC26A2 | N73101 | −1.5624 |
| 291623 | 64377 | Microphthalmia-associated transcription factor | MITF | N67822 | −1.6088 |
| 1845885 | 74393 | Transcribed sequence with strong similarity to protein pir: YRHU1 (H. sapiens) YRHU1 | | AI218117 | −1.70125 |
| | | monophenol monooxygenase | | | |
| 824358 | 37451 | Nuclear factor of activated T-cells, cytoplasmic, calcineurin-dependent 2 | NFATC2 | AA489681 | −1.8425 |
| 166652 | 75393 | ST3 beta-galactoside alpha-2,3-sialyltransferase 5 | ST3GAL5 | R88774 | −1.9226 |

Thirty four genes of interest were selected because of their possible involvement in the anticancer activity of α-TEA. The selected genes included apoptosis related genes (IER3, PHLDA1, TNFRSF12A, GADD45B, PMAIP1, STK17A and THBS1), signal transduction genes (RND3, RET, ABL2, ANXA1, TRIB1, ARHGEF2, RPS6KA3, RHOB, STRN3 and IL1RAP), genes involved in cell cycle (CCNL1, BTG1 and SESN2), cell adhesion and motility genes (SERPINE2), transcriptional regulators (ATF4, TES, MXD1, MYC, EGR1, PBX2, NFIL3, JUNB, CAMTA2, NFATC2 and SNAI2) as well as memebrane trafficing genes (SYTL2 and ST3GALS).

Cell Culture

MDA-MB-435 cells were cultured in MEM with Earle's balanced salts (Life Technologies, Inc., Grand Island, N.Y.) supplemented with 5% FBS (Hyclone Laboratories, Logan, Utah) plus 2 mM glutamine, 100 μg/ml streptomycin, 100 IU/ml penicillin, 1×(v/v) nonessential amino acids, 2×(v/v) MEM vitamins, and 1 mM sodium pyruvate (Sigma).

mRNA Isolation

Messenger RNA was isolated from the treated MDA-MB-435 cells (3×150 mm dishes) using the FastTrack 2.0 kit (Invitrogen) according to the manufacturer's instructions. First, 15 ml of lysis buffer was added into the cell pellets and mixed thoroughly. The cell lysates were incubated in 45° C. for 45 min with intermittent inversion. Then, 950 μl of 5 M NaCl stock solution was added and mixed thoroughly. Any remaining DNA was sheared by passing the lysates 3 times through a sterile plastic syringe fitted with a 21 gauge needle. Next, the calculated amount of oligo-dT cellulose was added to each sample and allowed to swell for 2 minutes. The tube was rocked gently in a horizontal position for 60-90 minutes at room temperature. The oligo-dT slurry was centrifuged at 4000 rpm for 5 minutes at room temperature, the supernatant was carefully removed from the resin bed. The oligo-dT cellulose was then resuspended in 20 ml of Binding Buffer by vortexing and centrifuged at 4000 rpm for 5 minutes at room temperature, the binding buffer was removed from the resin bed; this step was repeated with 10 ml of binding buffer. The oligo-dT cellulose was washed three times in 10 ml of Low Salt Wash Buffer and centrifuged. The resulting oligo-dT cellulose was resuspended in 800 μl of Low Salt Wash Buffer using a 1 ml pipette tip with a cut-end and quickly transferred to a spin column seated in a microcentrifuge tube. The column was centrifuged at 1000 rcf (about 3000 rpm on a typical microcentrifuge) for 10 seconds at room temperature and the flow-through liquid was discarded. The transfer and centrifugation steps were repeated until all of the oligo-dT cellulose was in the spin column. Then, the spin column containing the oligo-dT cellulose was placed into a clean microcentrifuge tube. The oligo-dT cellulose was resuspended in 200 μl of preheated Elution Buffer (65° C.) using a 200 μl cut-end pipette tip to gently swirl the cellulose, without puncturing the underlying spin column membrane. The column was allowed to stand for 2 minutes at room temperature and then centrifuged at 1000 rcf for 30 seconds at room temperature. This step was repeated with 200 μl of heated Elution Buffer. Then, 40 μl of 3 M sodium acetate and 1 ml of 95% ethanol were added into the combined 400 μl eluent which contained the mRNA and mixed thoroughly. The mRNA mixture was stored at −80° C. overnight. Thenext day, the mixture was thawed and centrifuged at 14,000 rpm in a microcentrifuge for 15 minutes at 4° C., the ethanol was then carefully removed from the mRNA pellet. The mRNA was washed with 70% ethanol once more and resuspended in 20 μl of Elution Buffer (heated as before).

cDNA Synthesis

Reverse transcription using the SuperScript II reverse transcriptase (Invitrogen) was carried out using 3 μg of mRNA as template and 5 μg of oligo-dT primer (5 μg/μl) (5′-TTT TTT TTT TTT TTT TTT TTV N-3′; SEQ ID NO:1) designed to anneal to the beginning of poly-A tails of the mRNA in the sample. The total volume of mRNA template and primer was 15.5 μl. The mixture was first incubated in 70° C. for 10 minutes and then chilled on ice for 10 minutes. Then the mRNA mixture was added into the enzyme mixture containing 1.9 μl of SuperScript II (200 U/μl; Invitrogen), 6 μl of 5×1st strand buffer, 0.3 μl of 1 M DTT, 5.1 μl diethylpyrocarbonate (DEPC) treated water, and 1.2 μl of 10 mM dNTP mix (PE Applied Biosystems, Foster City, Calif., USA). Reaction mixture (30 μl total volume) was incubated in 42° C. for 2 hours. cDNA was then purified using MinElute columns (Qiagen) and washed twice with 70% ethanol. In this process, in order to facilitate subsequent dye labeling process for microarray hybridization, amino-allyl modified dUTP was added to the RT reaction so the cDNA produced was randomly incorporated with the reactive group.

DNA/cDNA Labeling and Microarray Hybridization

The DNA/cDNA samples were all incorporated with aa-dUTP. This enabled indirect labeling of the DNA/cDNA samples by Cy-dyes containing NHS-ester group. DNA/cDNA samples were concentrated to 9 μl. 1 μl of fresh-made 1 M sodium bicarbonate (pH 9.0) was mixed into each sample. Cy-3 and Cy-5 mono-NHS-ester post-labeling reactive dyes (Amersham Biosciences) were resuspended using DMSO stored in desiccators. Then the dye and samples were mixed and incubated in the dark at room temperature. Typically, the samples of interest were labeled with Cy-5 and the control samples were labeled with Cy-3. Thus, the red to green ratio at each element served as a measure of the relative amount of certain species of DNA in the samples compared to the controls. After a 1 hour incubation, unincorporated dyes were washed out and the labeled DNA or cDNA samples were purified. Then the labeled samples of the desired pair were pooled together and added to the hybridization buffer which contained 5 μg human Cot-1 DNA, 10 μg polyA RNA, and 5 μg yeast tRNA (Invitrogen). The hybridization mixtures were then boiled for 2 minutes to denature the dsDNA, cooled down at room temperature for 30 minutes and applied to the post-processed microarrays. Hybridizations were performed in humidity chambers (Corning, Corning, N.Y., USA) at 65° C. for 16 hours. Slides were then washed, dried, and scanned using an Axon GenePix 4000 scanner (Axon Instruments, Union City, Calif.).

Array Scanning, Analyzing and Data Normalization

Arrays were scanned using GenePix 4000A/B scanner (Axon Instruments). The fluorescence intensities of the hybridized DNA/cDNA samples were measured at two wavelength channels, 532 nm (Cy3) and 635 nm (Cy5). Pre-made grids were then fitted onto the scanned images using GenePix 4.0 software (Axon Instruments). The grids contained spot information such as feature names and positions. Then images with grids were analyzed to determine the fluorescent intensities of each channel which represent the relative amount of DNA/cDNA in the samples. Result files, together with the image files, were uploaded to the Longhorn Array Database (Killion et al., 2003) for data processing. Normalization was carried out based on the assumption that the mean of all ratio values should be close to 1.0, because for any given experimental system relatively few genes were differentially expressed. In practice, first, a group of well-measured spots were defined by certain quality filters. Then the arithmetic mean of log-transformed Cy5/Cy3 ratios of these spots was calculated. This mean ratio was then used as the normalization factor and Cy5 channel measurements (net intensity) were divided by the normalization factor. Finally, normalized ratios were re-calculated and used for subsequent analysis.

### Example 2

### Arg is Involved in α-TEA Apoptotic Activity

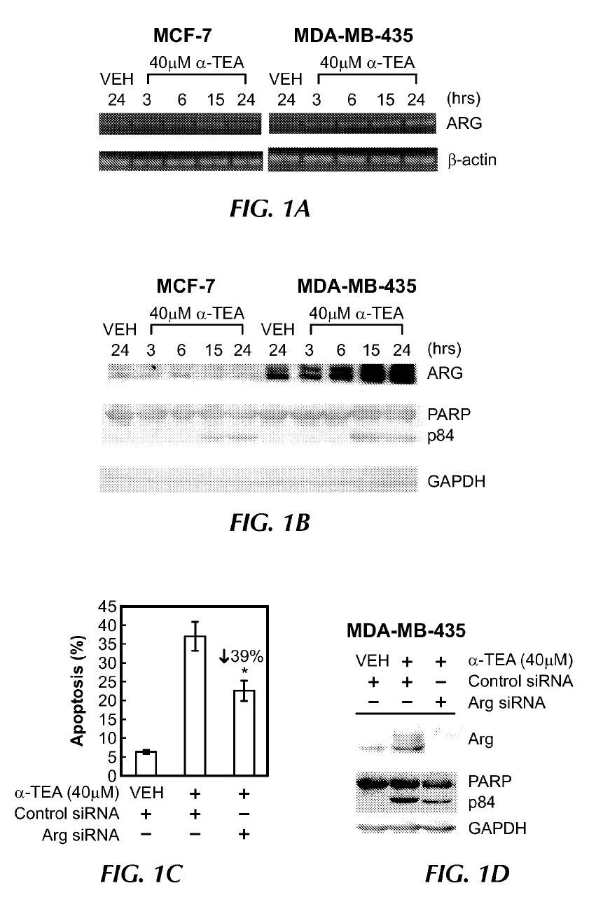

Arg (ABL2) expression data obtained from gene array analysis was further confirmed by RT-PCR and Western blot analyses. These further studies showed that mRNA and protein levels of Arg were up-regulated in α-TEA treated MDA-MB-435 human breast cancer cells, but not in MCF-7 cells (FIG. 1A-B). Unlike MDA-MB-435 cells, the MCF-7 cell line is an estrogen-receptor positive/estrogen responsive human breast cancer line (Klotz et al., 1995). However, both cell lines were induced to undergo apoptosis by α-TEA as assessed by PARP cleavage (FIG. 1B). Densitometric analyses showed peak Arg mRNA levels at 24 h post treatment to be 1.5-fold of the vehicle (VEH) control and peak Arg protein levels at 24 h to be 2.9-fold of the VEH control in MDA-MB-435 cells.

Furthermore, Arg siRNA significantly blocked α-TEA induced apoptosis (39% reduction) produced by treating the MDA-MB-435 cells with 40 μM of α-TEA for 15 h (FIG. 1C-D). As shown in FIG. 1D-top panel, Arg protein expression was almost completely abrogated by Arg siRNA. Reduction of Arg expression using siRNA in α-TEA treated MDA-MB-435 cells inhibited the p84 cleavage fragment of PARP, a marker for apoptosis, by 39% (FIG. 1C). GAPDH served as a loading control.

Western Analyses

Whole-cell protein extracts were prepared as described previously (Yu et al., 1999), and 50 μg of protein was loaded per lane, separated using SDS-PAGE on a 10-15% gel under reducing conditions, and electroblotted onto a nitrocellulose membrane (0.2 μM pore Optitran BA-S-supported nitrocellulose; Schleicher and Schuell, Keene, N.H.). Equal loading was verified using GAPDH antibody. Primary rabbit antibodies with specificity for PARP and primary goat antibody with specificity for Arg were purchased from Santa Cruz Biotechnology (Santa Cruz, Calif.). Horseradish peroxidase conjugated goat anti-rabbit or horseradish goat anti-mouse secondary antibody was purchased from Jackson Immunoresearch Laboratory (West Grove, Pa.). Horseradish peroxidase-conjugated donkey anti-goat serum was purchased from Santa Cruz Biotechnology. Immune complexes were visualized using enhanced chemiluminescence detection (Pierce Chemical Co., Rockford, Ill.). Fold differences in level of chemiluminescence were determined by densitometric analyses.

Reverse Transcription-Polymerase Chain Reaction

Total RNA was isolated from cells using RNeasy Protect Mini kit (Qiagen, Valencia, Calif.) according to manufacturer's instruction. Reverse transcription was performed as described above except using 15 μg of total RNA as template. Polymerase chain reaction (PCR) with Arg primers 30 cycles in volumes of 50 μl according to the manufacturer's protocol (Taq PCR Master Mix Kit; Qiagen, Valencia, Calif.). Primers used in the analyses were: Arg, forward (5′-CAG TGA TGC CTC CAC CTC AA-3′; SEQ ID NO:2) and reverse (5′-TTT CCC TCT CCC CTC AGA AAT-3′; SEQ ID NO:3) and for β-actin (loading control), forward (5′-GGC GGC ACC ACC ATG TAC CCT-3′; SEQ ID NO:4) and reverse (5′-AGG GGC CGG ACT CGT CAT ACT-3′; SEQ ID NO:5) (Invitrogen, Carlsbad, Calif.). The amplification reaction involved denaturation at 95° C. for 30 seconds, annealing at 52° C. for 30 seconds, and extension at 72° C. for 30 seconds (for Arg) using a PTC-225 thermal cycler (MJ Research, San Francisco, Calif.). The PCR products were resolved on 1% agarose gels and visualized by ethidium bromide staining.

Small Interfering RNA Knockdown of Arg

The double stranded small interfering RNA oligonucleotides specific for Arg and negative control siRNA (with no known homology to mammalian genes) were purchased from Ambion (siRNA ID #1478, Austin, Tex.). MDA-MB-435 cells (2×10⁵) were plated in 100 mm dishes. After overnight attachment, cells were transfected with siRNA duplex at a final concentration of 40 nM using LIPOFECTAMINE™ 2000 transfection reagent according to the manufacturer's instruction (Invitrogen, Carlsbad, Calif.). Culture media were replaced with normal growth media the next day. After another 24 to 48 h of incubation, the transfected cells were treated with ethanol (VEH control) or α-TEA for 15 h. The cells were collected, lysed and the lysates were analyzed by immunoblotting (Western blot).

### Example 3

### TSP-1 is Not Directly Involved in α-Tea Apoptotic Activity

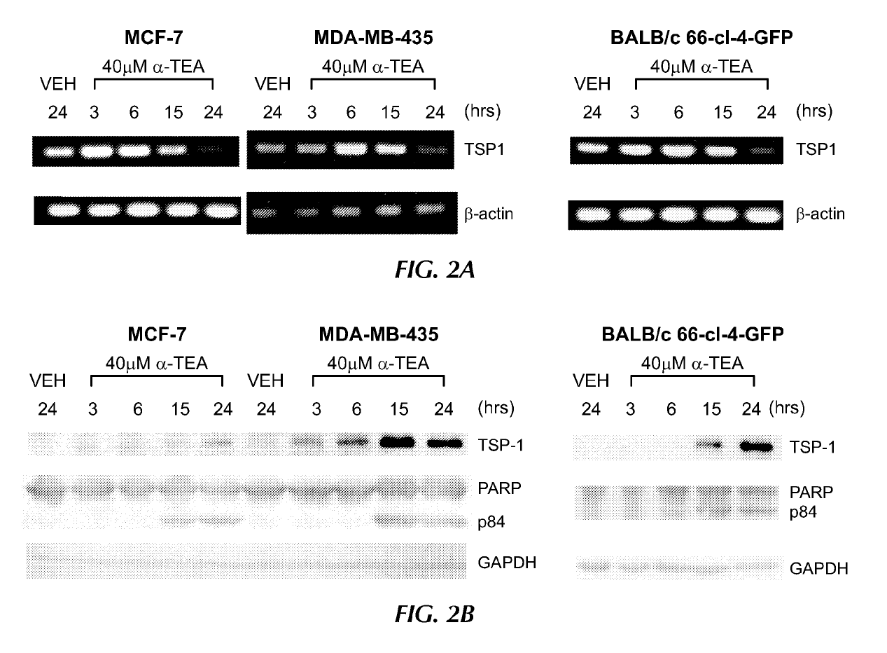

The TSP-1 gene was also shown to be up-regulated by α-TEA in the DNA array. Thus, this gene was further studied to determine if TSP-1 was relevant to the activity of α-TEA. mRNA levels of TSP-1 were shown to be up-regulated by RT-PCR as described example 1 using pimers forward (5′-AAC CGC ATT CCA GAG TCT GG-3′; SEQ ID NO:6) and reverse (5′-TTC ACC ACG TTG TTG TCA AGG GT-3′; SEQ ID NO:7) (FIG. 2A). Western blot analyses with a primary mouse antibody (Calbiochem (San Diego, Calif.)) was used to determine protein levels in treated versus untreated MDA-MB-435 and MCF-7 human breast cancer cells (FIG. 2B). 66c1-4-GFP cells also demonstrated increased levels of TSP-1 when treated with 40 μM α-TEA for 3 h, 6 h, 15 h and 24 h (FIG. 2A-B). 66c1-4-GFP cells are a mouse mammary tumor cell line originally derived from a spontaneous mammary tumor in a Balb/cfC3H mouse and later isolated as a 6-thioguanine-resistant clone. The cells were subsequently stably transfected with the enhanced GFP (Lawson et al., 2003). Densitometric analyses showed peak TSP-1 mRNA levels at 6 h α-TEA treatment were 1.8-fold of the vehicle (VEH) control and peak TSP-1 protein levels at 15 h were 4.7-fold of the VEH control in MDA-MB-435 cells. In MCF-7 cells, peak TSP-1 mRNA levels at 6 h were 1.3-fold of the VEH control and peak TSP-1 protein levels at 24 h were 2.4-fold of the VEH control. In 66c1-4-GFP cells, peak TSP-1 mRNA levels at 6 h were 2.4-fold of the VEH control and TSP-1 protein levels at 24 h were 7.8-fold of the VEH control.

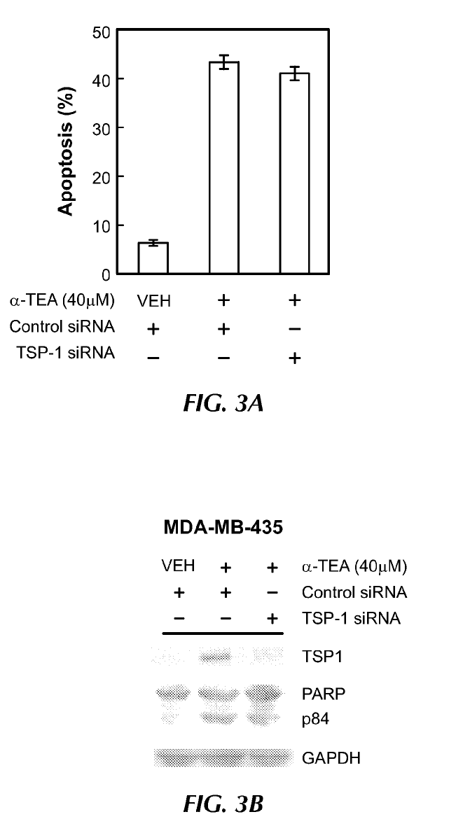

Further studies were undertaken to determine the effect of reduced TSP-1 protein levels on α-TEA induced apoptosis. For these studies a TSP-1 specific siRNA (The siRNA specific for human TSP-1 was purchased from Santa Cruz biotechnology (siRNA ID # sc36665, Santa Cruz, Calif.)) was used as described in example 1 for TSP-1 knock-down. However, blocking TSP-1 using TSP-1 siRNA in α-TEA treated MDA-MB-435 cells did not inhibit α-TEA-induced apoptosis or PARP cleavage (FIG. 3A-B), suggesting that TSP-1 is not directly involved in α-TEA-induced apoptosis in human breast cancer cells.

### Example 4

### α-TEA Decreased Levels of All Active, Phosphorylated Akt

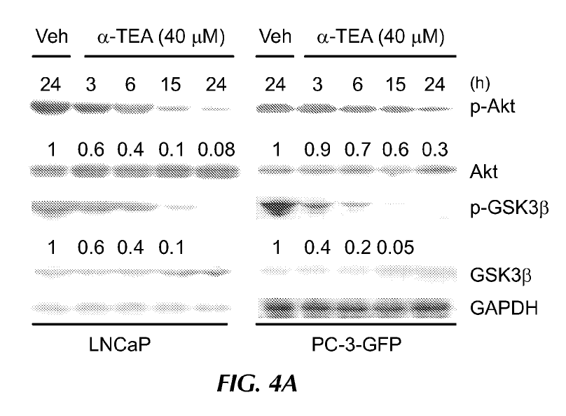

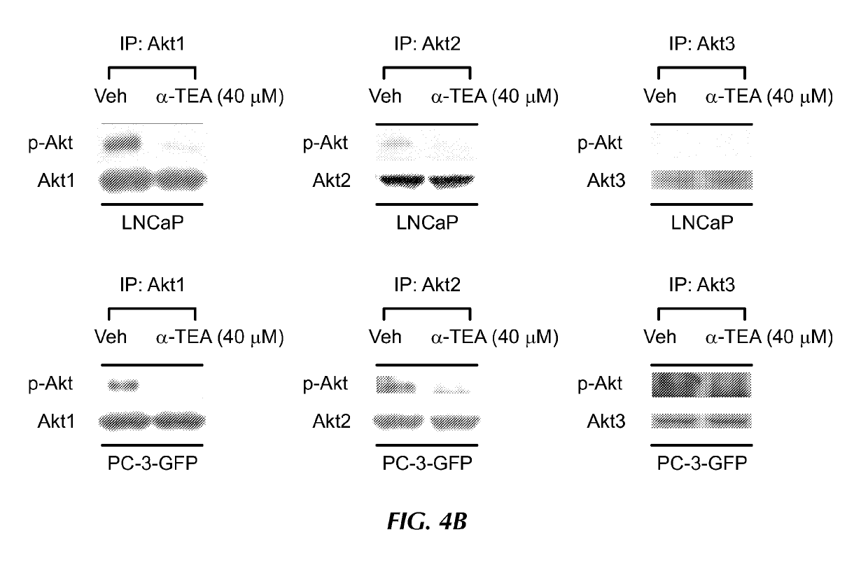

Previous studies have shown that α-TEA is an effective inducer of apoptosis in human prostate cancer cells (Anderson et al., 2004). Dose- and time-dependent pro-apoptotic effects of α-TEA in prostate cancer cells are mediated by Fas/FADD/caspase-8/tBid and Fas/Daxx/Ask1/JNK1/2/Bax, leading to activation of caspases-9 and -3. Thus, the Akt inhibiting activity of α-TEA was investigated in prostate cancer cells. Since phosphorylation of Akt at Ser 473 is required for its full activation (Cheng et al., 2005), we examined the phosphorylation status of Akt using an antibody that specifically recognizes Akt phosphorylated at Ser 473 in all three Akt isoforms (Upstate Cell Signaling Solutions (Charlottesville, Va.)). α-TEA decreased the levels of the phosphorylated forms of Akt. 24 h of α-TEA treatment reduced phosphorylation forms of Akt in LNCaP and PC-3-GFP cells by 90%, and 70% respectively (FIG. 4A). However, α-TEA treatment had no major effect on the level of Akt protein expression in either prostate cancer cell types at any time point (FIG. 4A). As verification that the decreases in phosphorylation status of Akt correlated with decreased kinase activity, the phosphorylation status of GSK3β, a downstream Akt target, was evaluated following α-TEA treatments. Decreases in the phosphorylation status of pGSK3β (at Ser 9) were detected with no corresponding decrease in total protein level, indicating that α-TEA treatments are reducing Akt activity (FIG. 4A). GAPDH levels were assayed with each immunoblot assay as a protein loading control.

Since Akt has three isoforms; namely, Akt1, Akt2, and Akt3, it was of interest to determine if α-TEA treatment was reducing the phosphorylated status of all three isoforms. Following α-TEA treatment the three Akt isoforms were immunoprecipitated with isoform specific antibodies, followed by western immunoblotting analyses using antibody specific for phospho-Akt. The protein levels of the isoforms were also determined. Except for Akt3 in LNCaP, all isoforms were constitutively activated in both cell types and α-TEA treatment markedly reduced phosphorylation levels of all three isoforms (FIG. 4B). Treatment diminished the phosphorylation levels in all three Akt isoforms in both LNCaP and PC-3-GFP cells.

Antibodies

Akt antibodies for the foregoing studies (Akt, Akt1, Akt2, Akt and phospho-Akt) as well as GSK3β and phospho-GSK3β antibodies were purchased from Cell Signaling Technology (Beverly, Mass.).

Immunoprecipitation

1×107 LNCaP or PC-3 3-GFP cells were treated with α-TEA and lysed in RIPA lysis buffer in the presence of protease inhibitors. 500 μg protein was incubated with anti-Akt1, Akt2, or Akt3 antibody at concentrations suggested by Cell Signaling Technology (namely: 1:500, 1:100, and 1:25, respectively) at 4° C. overnight, followed by the addition of 30 μl of protein G-agarose beads and an additional incubation at 4° C. for 2 h. Beads were washed 4 times with PBS and the proteins were released from the beads by boiling in Laemmli buffer for 5 min. Immunoprecipitated proteins were identified by SDS-PAGE followed by western immunoblot analyses.

Transient Transfection

Cells were plated at 2×10⁶ in 100 mm cell culture plates, cultured overnight and washed with serum free media before transfection. Plasmids (0.7 μg or 4.2 of μg) or empty vector (pcDNA3) were mixed with 50 μl or 300 μl serum free media and 4 μl or 24 μl PLUS reagent (Invitrogen, CA) and incubated for 15 min at room temperature. Next 2 μl or 12 μl of Lipofectamine reagent (Invitrogen, CA) in 50 μl or 300 μl serum free media were added and the mixture incubated for another 15 min before adding to the cells in 0.5 ml or 5 ml serum free media. Cells were incubated with transfection reagents for 3 h and the growth media containing 2×FBS was added to the cells. Transfected cells were cultured overnight before α-TEA treatment. Transfection efficiency was determined to be 70% using GFP expressing plasmid.

Evaluation of Apoptosis by DAPI Staining

Assessment of apoptosis based on nuclear morphology using 4′, 6-diamidino-2-phenylindole dihydrochloride (DAPI; Boehringer Mannheim Corp. Indianapolis, Ind.) has been described previously (Yu et al., 2003; Israel et al., 2000). Briefly, 1.5×10⁵/well LNCaP or PC-3-GFP cells were plated in 12-well tissue culture plates and cultured overnight to allow cells to attach. On the next day, cells were treated with various concentrations of α-TEA and incubated for various time periods. At the end of treatments, cells were collected, washed with PBS, stained with 25 μl of 2 μg/ml DAPI, and viewed under a fluorescent microscope (model ICM 405 with a model 487701 filter, Zeiss). Cells which contained clearly condensed chromatin or fragmented nuclei were scored as apoptotic cells. ≧500 cells were counted in each sample. Apoptotic data are presented as percentage of apoptotic cells±S.D. for three independent experiments.

### Example 5

### Active Akt Reduces α-TEA-Induced Apoptosis

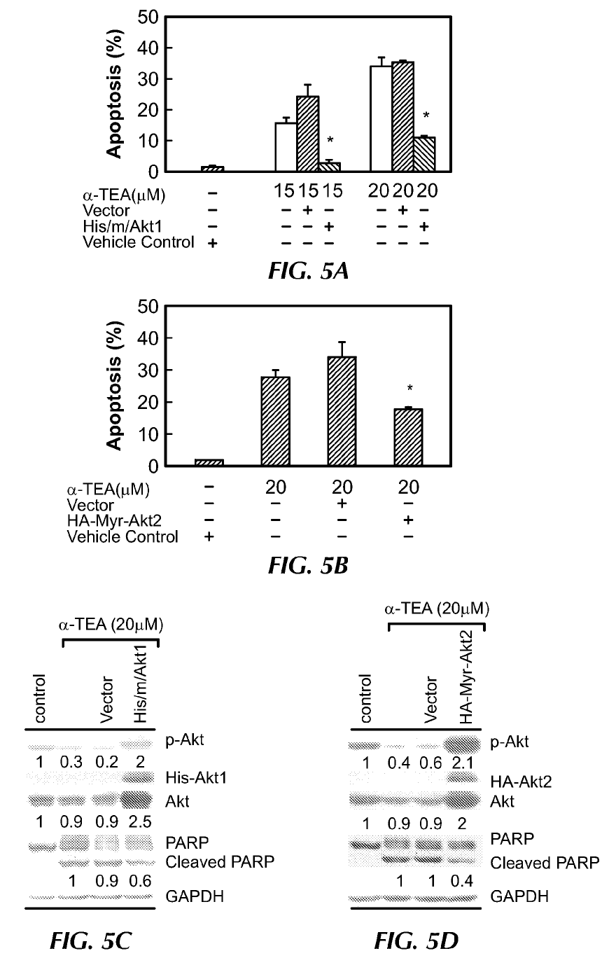

To address the biological relevance of Akt in α-TEA-induced apoptosis, constitutively active Akt1 or Akt2 expression plasmids (His/m/Akt1, HA-Myr-Akt2), or empty pcDNA3 vector control were transiently transfected into LNCaP cells. The His-tagged, myristalated constitutively active Akt1 plasmid (His/m/Akt1) was a kind gift of Dr. James Kehrer and is described in Tong et al., 2006. The hemagglutinin (HA)-tagged, myristalated constitutively active Akt2 plasmid (HA-Myr-Akt2) was kindly provided by Dr. Jin Q. Cheng and is described in Yuan et al., 2003. After α-TEA treatments, the percentage of apoptotic cells was significantly lower in cells expressing the constitutively active Akt1 (FIG. 5A; 21% decrease at 15 μM α-TEA and 24% decrease at 20 μM α-TEA) or Akt2 (FIG. 5B; 10% decrease at 20 μM α-TEA) compared to empty vector control. Success of transfections was confirmed by Western blot analyses of whole cell lysates which showed transfected cells exhibited increased levels of phosphorylated Akt (FIGS. 5C and 5D, first panel), confirmed expression of ectopically expressed Akt via detection of fused antigenic tags (FIGS. 5C and 5D, second panel, His and HA tags respectively), and by increased levels of total Akt (FIGS. 5C and 5D, third panel). Detection of reduced levels of PARP cleavage (FIGS. 5C and 5D, fourth and fifth panels) also confirmed that ectopic expression of active Akt blocked α-TEA-induced apoptosis.

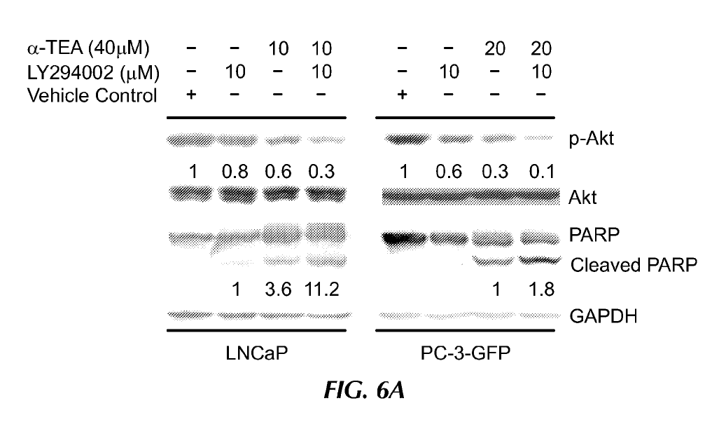

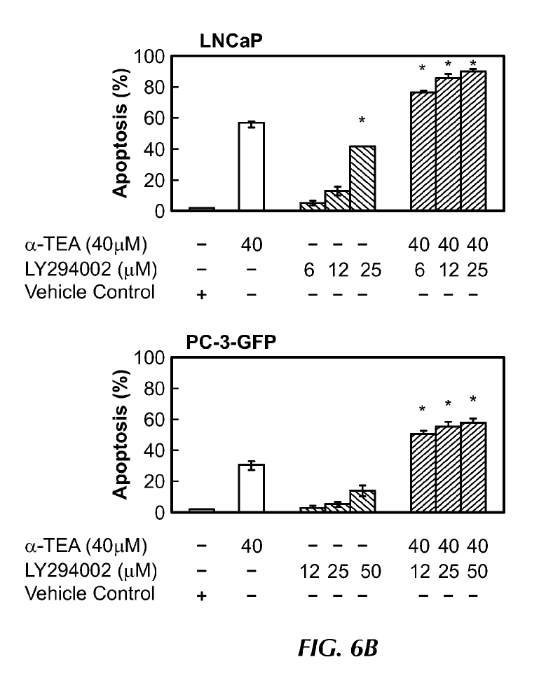

Since Akt is activated, at least in part, by PI3K, we were interested to see if blockage of PI3K activity using the PI3K inhibitor LY294002 (Calbiochem-Novabiochem Corp. (San Diego, Calif.)) (Vlahos et al., 1994) would augment the apoptotic response induced by α-TEA. Although treatment of cells with either LY294002 or α-TEA singly inhibited the levels of endogenously phosphorylated Akt in both cell lines without changing total Akt protein levels (FIG. 6A) by 20 and 40% in LNCaP and 60% and 70% in PC-3-GFP cells, respectively, combination treatment with LY294002+α-TEA produced the most effective blockage, reducing levels of phosphorylated Akt by 70% and 90%. Western immunoblot analyses showed that the combination treatment produced a higher degree of PARP cleavage than the individual treatments (FIG. 6A, third and fourth lanes from the top). Quantitative analyses of apoptosis was performed as described supra and (FIG. 6B) showed that blockage of PI3K with LY294002 triggered apoptosis in both cell types, with the combination treatment of LY294002+α-TEA producing significantly higher (P<0.001) levels of apoptosis in comparison to either α-TEA or LY294002 treatment alone. Furthermore, these studies confirmed earlier reports that LNCaP prostate cancer cells are more dependent on PI3K-induced survival signals than PC-3 cells (Lin et al., 1999), since LNCaP cells showed a dose-dependent induction of apoptosis of 4.9, 13 and 41%, following treatments with 6, 12 and 25 μM LY294002. In contrast, PC-3-GFP cells exhibited only 6.2% apoptotic cells following 25 μM LY294002 and only approximately 15% apoptotic cells when treated with twice that concentration; namely 50 μM LY294002.

### Example 6

### The Role of FOXO1 in α-TEA Activity

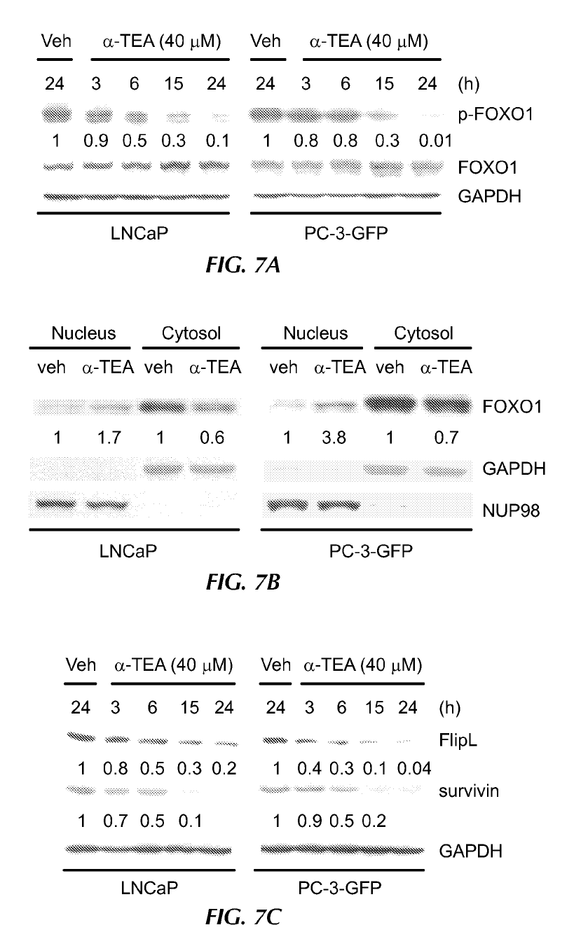

The transcription factor FOXO1 is a downstream substrate of Akt in which phosphorylation by Akt prevents its pro-apoptotic actions (Woods et al., 2002). Since previous studies of α-TEA's mechanism of action in inducing apoptosis in cancer cells show a role for Fas signaling (Shun et al., 2004) and since FOXO1 has been proposed to be a transcriptional regulator of FasL (Ciechomska et al., 2003), it was of interest to investigate the effects of α-TEA on FOXO1 phosphorylation status and cellular location. As shown in FIG. 7A, phospho-FOXO1 levels, but not total protein levels, were reduced in a time-dependent manner following α-TEA treatment of both LNCaP and PC-3-GFP cells. FOXO1 is normally localized in the nucleus (Woods et al., 2002) and phosphorylation of FOXO1 by Akt at Ser 256 promotes the relocation of FOXO1 from the nucleus to the cytosol, thereby inhibiting its transcriptional activity (Greer et al., 2005). As expected, control LNCaP and PC-3-GFP cells had high levels of FOXO1 in the cytosol reflecting the constitutively activated state of Akt in these cells (FIG. 7B). Also as expected, following treatment with α-TEA (40 μM for 24 h), both cell types showed an increase in FOXO1 levels in the nucleus (FIG. 7B).

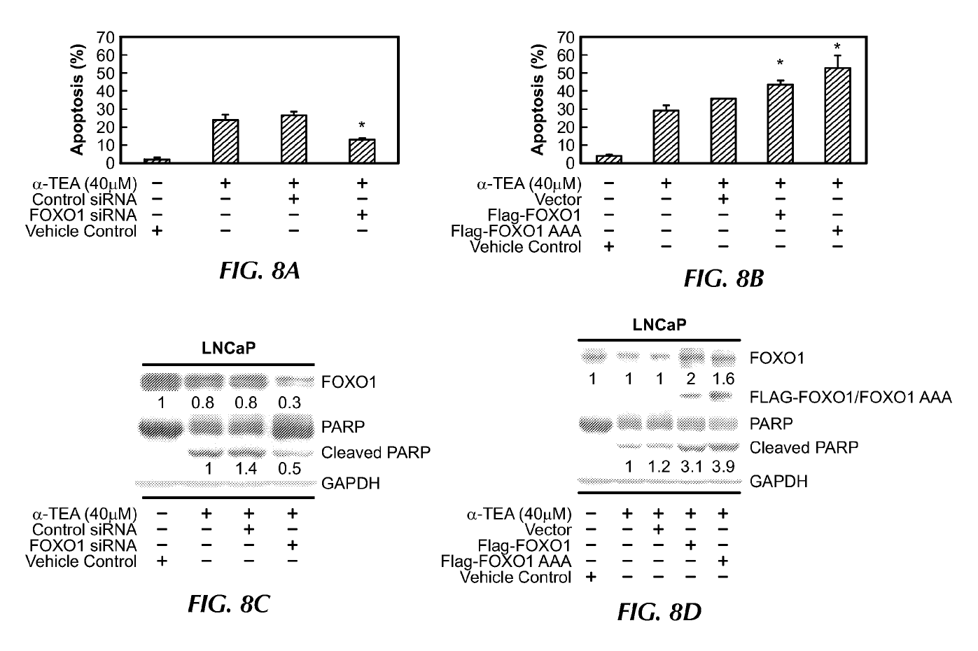

In order to address the question of whether or not FOXO1 expression contributes to α-TEA induced apoptosis, FOXO1 expression was knocked down in LNCaP cells using siRNA, and effects on α-TEA-induced apoptosis was determined (FIG. 8A). Introducing FOXO1 siRNA into LNCaP cells significantly inhibited α-TEA induced apoptosis by 15% (FIG. 8A). Western immunoblot analyses confirmed the siRNA-induced reductions in FOXO1 protein level in LNCaP cells and confirmed blockage of α-TEA-induced apoptosis by a 50% decrease in PARP cleavage (FIG. 8B). On the other hand, overexpression of Flag-tagged wild type FOXO1 or Flag-tagged constitutively active FOXO1 (Flag-FOXO1 AAA) increased α-TEA-induced apoptosis significantly (FIG. 8C; P<0.009). Western immunoblot analyses confirmed elevated levels of Flag Flag-tagged wild type FOXO1 or constitutively active Flag-FOXO1 in the transfected LNCaP cells and showed increased PARP cleavage in comparison to empty vector control transfected cells (FIG. 8D; 160%, and 230%, respectively). Taken together, these data document a role for FOXO1 in α-TEA-induced apoptosis in human prostate cancer cells.

Plasmids and Antibodies

Plasmids encoding wild type FOXO1 (Flag-tagged FOXO1) and constitutively active FOXO1 (Flag-tagged FOXO1AAA) were generous gifts from Dr. Kun-Liang Guan (Tang et al., 1999). FOXO1 and phospho-FOXO1 antibodies were purchased from Cell Signaling Technology (Beverly, Mass.).

Cell Fractionation

Preparation of cytoplasmic and nuclear extracts for the foregoing experiments was carried-out using methods that are well known in the art. Breifly, 1×10⁷ cells were treated, harvested and resuspended in 200 μl of extraction buffer (10 mM HEPES, pH 7.9, 1.5 mM Mg Cl₂, 10 mM KCl) for 10 min on ice. Next, cells were homogenized by passage through a 25-guage needle. Cell homogenates were centrifuged at 2,000 rpm for 5 min. Supernatants (cytosolic fraction) were centrifuged 3 times at 2,000 rpm for 5 min, changing tubes after each centrifugation. Pellets (nuclear fraction) were washed 3 times in PBS, and lysed in RIPA buffer (1×PBS, pH7.4, 1% NP40, 0.5% sodium deoxycholate, 0.1% SDS, 1 μg/ml leupeptin, 1 μg/ml aprotinin, 1 mM DTT, 2 mM sodium orthovanadate, 1 μg/ml phenylmethylsulfonyl fluoride). 50 μg protein samples were resolved on 10% or 15% SDS-PAGE and subject to Western blot as described supra.

RNA Interference

FOXO1 siRNA used in the study was purchased from Santa Cruz (Santa Cruz, Calif.). A scrambled RNA duplex that does not target any known FOXO/FKHR genes was used as the negative control. Transfection of human prostate cancer cells with FOXO1 siRNA or negative control siRNA was performed in 100 mm cell culture dishes at a density of 2×10⁶ cells/dish using Lipofectamine 2000 (Invitrogen, Carlsbad, Calif.) and 300 pmol of siRNA duplex, resulting in a final siRNA concentration of 30 nM. siRNA transfected cells were incubated for 48-72 h prior to α-TEA treatment.

### Example 7

### α-TEA Downregulates Flip and Survivin

It was of interest to know if α-TEA had a downregulatory effect on Flip and survivin in human prostate cancer cells. Immunoblot sudies showed that protein levels of FlipL and survivin were downregulated by α-TEA treatment in both LNCaP and PC-3-GFP cells in a time-dependent manner (FIG. 7C). Antibodies to human Flip and survivin used in these experiments were purchased from Santa Cruz Biotechnology (Santa Cruz, Calif.).

### Example 8

### NOXA is Regulated by α-TEA in Human Breast Cancer Cells

In order to identify new signaling molecules modulated by α-TEA, DNA microarray experiments were carried out using MDA-MB-435 human breast cancer cells treated with 40 μM of α-TEA for 12 h as described supra. Genes with a log₂-transformed treatment/control ratio of ≧1 or ≦−1 were considered to be up-regulated or down-regulated by α-TEA, respectively. Based on the above analyses, NOXA was identified to be responsive to α-TEA.

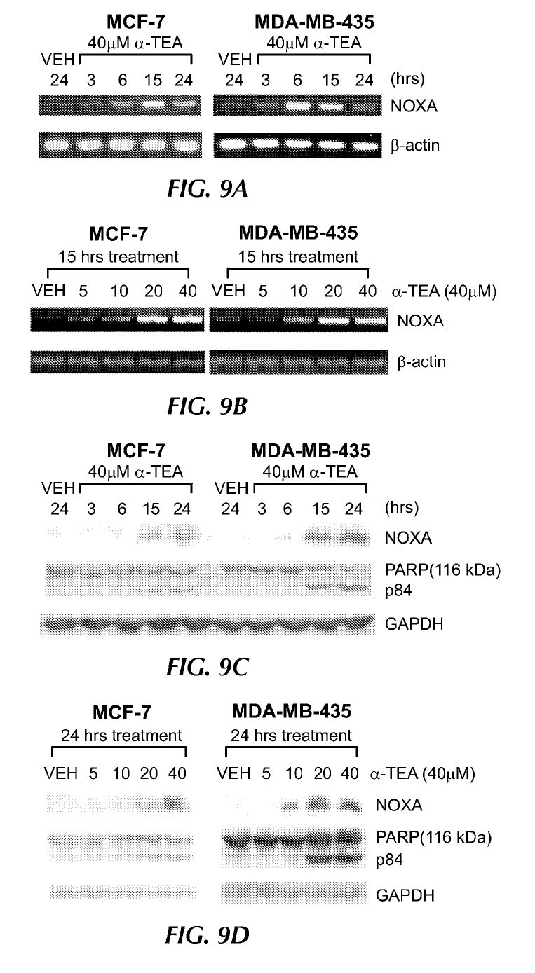

To confirm the microarray data, semi-quantative RT-PCR and Western blot analyses were carried out to measure the change in mRNA and protein level of NOXA using estrogen nonresponsive MDA-MB-435 and estrogen responsive MCF-7 human breast cancer cells. Assays were preformed as describide above using NOXA specific antibodies (purchased from Santa Cruz Biotechnology (Santa Cruz, Calif.)) and NOXA sepcific PCR primers (forward (5′-CGT GTG TAG TTG GCA TCT CC-3′) and reverse (5′-GCC CCA AGT AAC CCT CCT AT-3′)). In MCF-7 cells, α-TEA induced NOXA mRNA starting at 3 h, peaking at 15 h (FIG. 9A, left top panel). In MDA-MB-435 cells, α-TEA induced NOXA mRNA starting at 3 h, peaking at 6 h (FIG. 9A, right top panel). In both cell lines, protein levels of NOXA were increased at 15 and 24 h after α-TEA treatments (FIG. 9C, top panel). Densitometric analyses showed peak NOXA mRNA levels at 6 h to be 2.6-fold higher than the vehicle (VEH) control and NOXA protein levels at 24 h to be 16.8-fold higher than the VEH control in MDA-MB-435 cells. In MCF-7 cells, peak NOXA mRNA levels at 15 h were 1.5-fold higher than the VEH control and NOXA protein levels at 24 h were 7.6-fold higher than the VEH control. Upregulation of NOXA protein levels is correlated with PARP cleavage which is an indicator of cells undergoing apoptosis (Wolf and Green, 1999) (FIG. 9C, second panel). GAPDH levels served to help verify lane loads (FIG. 9C, bottom panel). Treatment of MCF-7 and MDA-MB-435 cells for 3-24 h with 40 μM α-TEA (FIGS. 9A and C) or with different concentrations of α-TEA (FIGS. 9B and D) showed elevated levels of NOXA mRNA and NOXA protein to be both time- and dose-dependent in both cell lines. NOXA protein levels were also assessed in other cell lines including MDA-MB-231 and T47D human breast cancer cell lines, immortalized, but nontumorigenic MCF-10A breast epithelial cells as well as human normal mammary epithelial cell line (HMEC) (Table 3). The ability of α-TEA to induce NOXA and induce apoptosis plus the p53 status based on the literature is summarized (Table 3). NOXA was induced in all breast cancer cell lines in which α-TEA induces apoptosis, and it was not induced in T47D human breast cancer cells and HMEC cells in which α-TEA does not induce apoptosis (Anderson et al., 2004). NOXA induction by α-TEA does not correlate with p53 status, but rather is p53-independent (Table 3).

TABLE 3

| | Ability of α- | | Ability of α-TEA |
|---|---|---|---|
| | TEA to induce | | to induce NOXA |
| Cell lines | apoptosis^{a} | p53 status^{b} | expression^{c} |
| MCF-7 | ++ | W (Fan et al., 2002) | Yes |
| MDA-MB-435 | ++ | M (Fan et al., 2002) | Yes |
| MDA-MB-231 | ++ | M (Fan et al., 2002) | Yes |
| T47D | − | M (Fanayan et al., | No |
| | | 2000) | |
| HMEC | − | W (Seewaldt et al., | No |
| | | 2001) | |
| MCF-10A | +/− | W (Levesque et al., | Yes |
| | | 2005) | |

### Example 9

### NOXA siRNA Blocks α-TEA-Induced Apoptosis

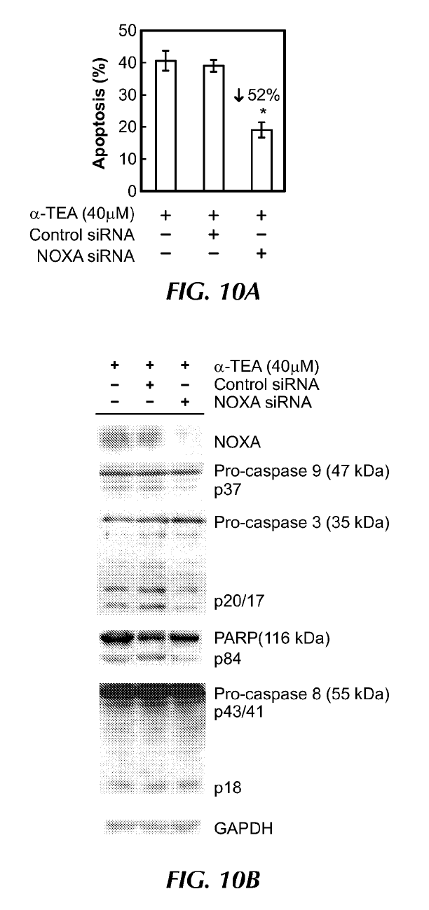

To address the role of NOXA in α-TEA-induced apoptosis, MDA-MB-435 cells were transiently transfected with NOXA siRNA. For NOXA dsRNA was sense (5′-GCU AUU UUA CCA UCU GGU Att-3′) and antisense, (5′-UAC CAG AUG GUA AAA UAG Ctg-3′) while control dsRNA was a standard control commercially available from Ambion (Austin, Tex.) that has no known homology to mammalian sequences. Results showed that NOXA siRNA significantly blocked α-TEA induced apoptosis produced by treating the cells with 40 μM of α-TEA for 15 h by 52% (FIG. 10A). As shown in FIG. 10B-top panel, NOXA was almost completely knocked down by NOXA siRNA.

Blocking NOXA using siRNA in α-TEA treated MDA-MB-435 cells inhibited NOXA (FIG. 10B, top panel), inhibited the p37 cleavage fragment of caspase 9 (FIG. 10B, second panel), inhibited the p20/17 cleavage fragments of caspase 3 (FIG. 10B, third panel), and inhibited the PARP cleavage fragment p84 (FIG. 10B, fourth panel). As shown in the fifth panel of FIG. 10B, caspase 8 cleavage product p18 was not inhibited. Densitometric analyses of these data showed the cleavage of caspase 9, caspase 3, and PARP was reduced by 47%, 32%, and 50% respectively, using NOXA siRNA in comparison to control siRNA in α-TEA treated MDA-MB-435 cells. Antibodies used in the foregoing Western blot analyses were purchased from Santa Cruz Biotechnology (Santa Cruz, Calif.).

### Example 10

### α-TEA Mediates NOXA Via JNK

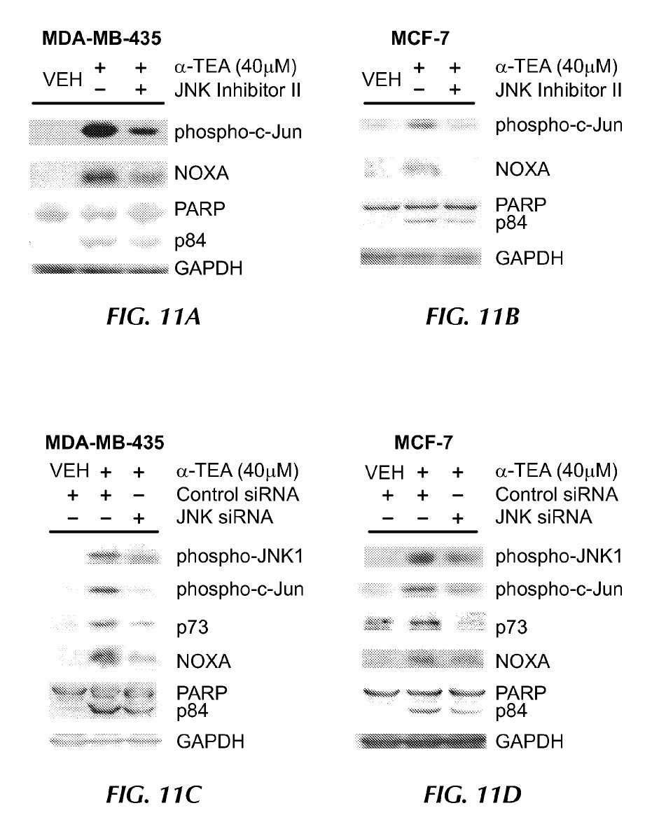

Because JNK has been shown to be involved in α-TEA-induced apoptosis in MDA-MB-435 cells, and because inhibition of JNK with a dominant-negative construct blocked mitochondria-dependent apoptotic events in vitamine E succinate treated MDA-MB-435 cells (Shun et al., 2004; Yu et al., 1998), the role of JNK in α-TEA-induced NOXA expression was investigated. When MDA-MB-435 cells were treated with JNK-inhibitor II for 2 h before treatment with 40 μM α-TEA, analyses of whole cell extracts showed that the JNK inhibitor reduced the ability of α-TEA to induce c-Jun phosphorylation (FIG. 11A, top panel), level of NOXA expression (FIG. 11A, second panel), and level of PARP cleavage (FIG. 11A, third panel). Densitometric analyses showed that the levels of phosphorylated c-Jun, NOXA, and PARP cleavage were reduced by 38%, 41% and 34%, respectively, in comparison to vehicle control. Similar results were also observed in α-TEA treated MCF-7 cells in the presence of JNK inhibitor II (7.5 μM of JNK inhibitor II (Calbiochem-Novabiochem Corp. San Diego, Calif.) vs. DMSO control). Specifically, 68% reduction in the level of phosphorylated c-Jun (FIG. 11B, top panel), almost 100% reduction in NOXA expression (FIG. 11B, second panel), and 29% reduction in the level of PARP cleavage (FIG. 11B, third panel).

JNK siRNA was used to determine if JNK was involved in α-TEA regulation of NOXA expression. MDA-MB-435 and MCF-7 cells were transiently transfected with siRNA specific for JNK1/2 (JNK1/2 sense (5′-AAA GAA UGU CCU ACC UUC Utt-3′), JNK1/2 antisense (5′AGA AGG UAG GAC AUU CUU Utt-3′)). The cells were then treated with α-TEA for 15 h, or 20 h, respectively. Since previous studies showed that the activation of JNK1 isoform is involved in α-TEA-induced apoptosis, the phosphorylation of JNK1 was detected using Western immunoblot analyses. Data show that in MDA-MB-435 cells, JNK siRNA blocked JNK1 phosphorylation by 45% (FIG. 11C, first panel), blocked c-Jun phosphorylation by 85% (FIG. 11C, second panel), reduced protein levels of full length p73 by 73% (FIG. 11C, third panel), reduced protein levels of NOXA protein by 76% (FIG. 11C, fourth panel), and reduced level of PARP cleavage by 20% (FIG. 3C, fifth panel). Likewise in MCF-7 cells JNK siRNA blocked JNK1 phosphorylation by 26% (FIG. 11D, first panel), blocked c-Jun phosphorylation by 36% (FIG. 1D, second panel), reduced protein levels of full length p73 by 66% (FIG. 1D, third panel), reduced protein levels of NOXA by 14% (FIG. 1D, fourth panel), and reduced level of PARP cleavage by 44% (FIG. 11D, fifth panel). Anti-p73 antibodies used in the foregoing experiment were purchases from IMGENEX (San Diego, Calif.).

### Example 11

### α-TEA Induces NOXA Expression Via a p73-Dependent Pathway

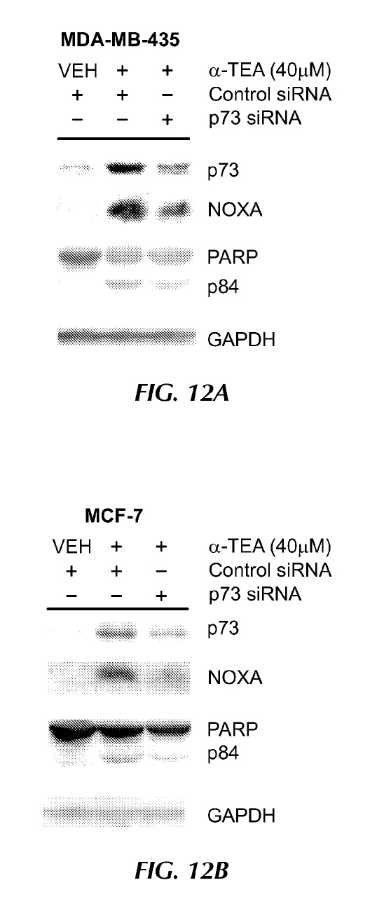

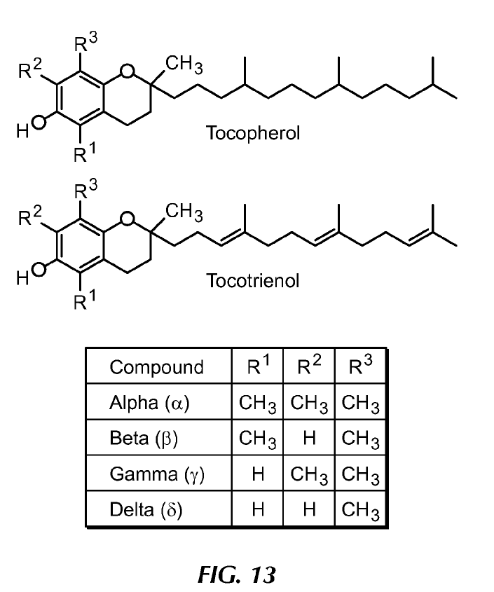

Finding that inhibition of JNK reduced the expression of full length p73 as well as NOXA in both cell lines (FIG. 11), it was of interest to see if p73 was playing a role in NOXA expression. Since other isoforms of p73 were either not detectable in the cell lysates, or the changes in the other isoforms were not observed, full length p73 (TAp73a) appeared to play a dominant role in the anticancer activity of α-TEA. p73 siRNA (p73 sense (5′-CGG AUU CCA GCA UGG ACG Utt-3′), p73 antisense (5′-ACG UCC AUG CUG GAA UCC Gtt-3′)) was transiently transfected into p53-deficient MDA-MB-435 cells and MCF-7 cells that express wild type p53 but do not readily undergo p53-dependent apoptosis (Fan et al., 1995). Analyses of cellular extracts derived from MDA-MB-435 and MCF-7 cells treated with 40 μM of α-TEA for 15 h and 20 h, respectively, showed that p73 siRNA blocked full length p73 protein expression (FIGS. 12A and B, first panel), reduced NOXA expression (FIGS. 12A and B, second panel), and reduced PARP cleavage (FIGS. 12A and B, third panel). Densitometric analyses of data from MDA-MB-435 and MCF-7 cells showed that p73 siRNA reduced full length p73 protein levels by 54% and 61%, blocked NOXA expression by 53% and 42%, and blocked PARP cleavage by 35% and 48%, respectively.

Primary mouse antibody specific for p73 was purchased from IMGENEX (San Diego, Calif.). Primary mouse antibody specific for caspase 8 was purchased from Cell Signaling Technology (Beverly, Mass.). Horseradish peroxidase conjugated goat anti-rabbit or goat anti-mouse secondary antibodies were purchased from Jackson Immunoresearch Laboratory (West Grove, Pa.). Horseradish peroxidase-conjugated donkey anti-bovine serum was purchased from Santa Cruz Biotechnology. Immune complexes were visualized using enhanced chemiluminescence detection (Pierce Chemical Co., Rockford, Ill.). Fold differences in level of chemiluminescence were determined by densitometric analyses.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,376,110
U.S. Pat. No. 4,816,567
U.S. Pat. No. 4,988,682
U.S. Pat. No. 5,053,399
U.S. Pat. No. 5,958,773
U.S. Pat. No. 6,043,090
U.S. Pat. No. 6,124,272
U.S. Pat. No. 6,187,586
U.S. Pat. No. 6,245,754
U.S. Pat. No. 6,417,223
U.S. Pat. No. 6,703,384
U.S. Pat. No. 6,770,672
U.S. Pat. No. 6,949,537
U.S. Publn. 20030158212
U.S. Publn. 20030149074
U.S. Publn. 20030236301
Amaravadi et al., J. Clin. Invest., 115(10):2618-2624, 2005.
Anderson et al., Cancer Res., 15:64(12):4263-4269, 2004.
Anderson et al., Exp. Biol. Med., 229(11):1169-1176, 2004.
Bhandari et al., Eur. J. Cancer, 41(6):941-953, 2005.
Birkenkamp and Coffer, Biochem. Soc. Trans., 31(1):292-297, 2003.
Cao et al., J. Biot. Chem., 276:11465-11468, 2001.
Cao et al., J. Biol. Chem., 278:12961-12967, 2003.
Ceichomaska et al., Oncogene, 22:7617-7627, 2003.
Cheng et al., Oncogene, 24(50):7482-7492, 2005.
David et al., Biochemistry, 13:1014, 1974.
Fan and Cheman, Br. J. Cancer, 87:1019-1026, 2002.
Fan et al., Cancer Res., 55:1649-1654, 1995.
Fanayan et al., J. Biol. Chem., 275:39146-39151, 2000.
Fresno Vara et al., Cancer Treat. Rev., 30(2):193-204, 2004.
Goding, In: Monoclonal Antibodies: Principles and Practice, 2d ed., Academic Press, Orlando, Fla., pp 60-61, 71-74, 1986.
Greer et al., Oncogene, 24(50):7410-7425, 2005.
Hennessy et al., Nat. Rev. Drug Discov., 4(12):988-1002, 2005.
Hunter et al., Nature, 144:945, 1962.
Israel et al., Nutr. Cancer, 36(1):90-100, 2000.
Jemal et al., CA Cancer J. Clin., 56:106-130, 2006.
Jope and Johnson, Trends. Biochem. Sci., 29:95-102, 2004.
Killion et al., BMC Bioinformatics, 4:32, 2003.
Klotz et al., Biochem. Biophys. Res. Commun., 210:609-615, 1995.
Kohler and Milstein, Nature, 256:495-497, 1975.
Lawler, J. Cell. Mol. Med., 6:1-12, 2002.
Lawson et al., Cancer Chemother. Pharmacol., 54(5):421-431, 2004.
Lawson et al., Exp. Biol. Med., 229(9):954-963, 2004.
Lawson et al., Mol. Cancer Ther., 2:437-444, 2003.
Levesque et al., Oncogene, 24:3786-3796, 2005.
Li et al., Prostate Cancer Prostatic Dis., 8:108-118, 2005.
Lin et al., Cancer Res., 59(12):2891-2897, 1999.
Linseman et al., J. Neurosci., 24:9993-10002, 2004.
Majumder and Sellers, Oncogen., 24:7465-7474, 2005.
Mandal et al., Oral Oncol., 42:430-439, 2006.
Martelli et al., Leukemia, 17(9):1794-1805, 2003.
Morrison et al., Proc. Natl. Acad. Sci. USA, 81(21):6851-6855, 1984.
Mulholland et al., Oncogene, 25:329-337, 2006.
Munson and Pollard, Anal. Biochem., 107:220, 1980.
Nygren, J. Histochem. Cytochem., 30(5):407-412, 1982.
Pain et al., J. Immunol. Meth., 40:219, 1981.
Pastorino et al., Cancer Res., 65(22):10545-10554, 2005.
Paez and Sellers, Cancer Treat Res, 115:145-67, 2003.
Price et al., Cancer Res., 50:717-721, 1990.
Remington's Pharmaceutical Sciences, 18th Ed. Mack Printing Company, pp. 1289-1329, 1990.
Ren et al., Biochim. Biophys. Acta., 1765:178-188, 2006.
Seewaldt et al., Cancer Res., 61:616-624, 2001.
Shimada et al., Mol. Carcinog., 39(1)1-9, 2004.
Shun et al., Nutr. Cancer, 48(1):95-105, 2004.
Sid et al., Crit. Rev. Oncol. Hematol., 49:245-258, 2004.
Song et al., J. Cell Mol. Med., 9(1):59-71, 2005.
Tang et al., J. Biol. Chem., 274(24):16741-16746, 1999.
Tang et al., 34th Annual Meeting Society of Gynecologic Oncologists: abstract 213, 2003
Tong et al., Lung Cancer, 52(1):117-124, 2006.
Vlahos et al., J. Biol. Chem., 269:5241-5248, 1994.
Wolf and Green, J. Biol. Chem., 274:20049-20052, 1999.
Woods and Rena, Biochem. Soc. Trans., 30(4):391-397, 2002.
Xue et al., Proc. Natl. Acad. Sci. USA, 99:6925-6930, 2002.
Yu et al., Cancer Res., 63(10):2483-91, 2003.
Yu et al., Cancer Res., 59:953-961, 1999.
Yu et al., Mol. Carcinog., 22:247-257, 1998.
Yuan et al., J. Biol. Chem., 278(26):23432-23440, 2003.
Zhang et al., Breast Cancer Res. Treat., 87:111-121, 2004.
Zola, In: Monoclonal Antibodies: A Manual of Techniques, CRC Press, Inc., pp. 147-158, 1987.

1. A method for treating a cancer patient wherein the patient comprises a Arg, p73, NOXA or FOXO1 positive cancer the method comprising administering an effective amount of a chroman ring derivavtives compound.

2. The method according to claim 1, wherein the cancer patient comprises a cancer cell that overexpresses a Arg, p73, NOXA or FOXO1 gene relative to a normal cell.

3. The method according to claim 1, wherein the cancer patient comprises a Arg, p73, NOXA and FOXO1 positive cancer cell.

4. (canceled)

5. The method of claim 1, wherein the chroman ring derivative compound is an alpha, beta, gamma or delta tocopherol or tocotrienol.

6. (canceled)

7. The method of claim 1, wherein the chroman ring derivavtive compound is 2,5,7,8-Tetramethyl-(2R-(4R,8R,12-trimethyltridecyl)chroman-6-yloxy)acetic acid (α-TEA), 2,5,7,8-Tetramethyl-(2R-(4R,8R,12-trimethyltridecyl)chroman-6-yloxy)propionic acid, 2,5,7,8-Tetramethyl-(2R-(4R,8R,12-trimethyltridecyl)chroman-6-yloxy)butyric acid, 2,5,8-Trimethyl-(2R-(4R,8R,12-trimethyltridecyl)chroman-6-yloxy)acetic acid, 2,7,8-Trimethyl-(2R-(4R,8R,12-trimethyltridecyl)chroman-6-yloxy)acetic acid, 2,8-Dimethyl-(2R-(4R,8R,12-trimethyltridecyl)chroman-6-yloxy)acetic acid, 2-(N,N-(carboxymethyl)-2-(2,5,7,8-tetramethyl-(2R-(4R,8R,12-trimethyltridecyl)chroman-6-yloxy)acetic acid, 2,5,7,8-Tetramethyl-(2RS-(4RS,8RS,12-trimethyltridecyl)chroman-6-yloxy)acetic acid, 2,5,7,8-Tetramethyl-2R-(2RS,6RS,10-trimethylundecyl)chroman-6-yloxy)acetic acid, 3-(2,5,7,8-Tetramethyl-(2R-(4R,8,12-trimethyltridecyl)chroman-6-yloxy)propy 1-1-ammonium chloride, 2,5,7,8-Tetramethyl-(2R-(4R,8R,12-trimethyltridecyl)chroman-3-ene-6-yloxy)acetic acid, 2-(2,5,7,8-Tetramethyl-(2R-(4R,8,12-trimethyltridecyl)chroman-6-yloxy)triethylammonium sulfate, 6-(2,5,7,8-Tetramethyl-(2R-(4R,8,12-trimethyltridecyl)chroman)acetic acid, 2,5,7,8-Tetramethyl-(2R-(heptadecyl)chroman-6-yloxy)acetic acid, 2,5,7,8-Tetramethyl-2R-(4,8,-dimethyl-1,3,7 E:Z Nonotrien)chroman-6-yloxy)acetic acid, E.Z, RS, RS, RS-(Phytyltrimethylbenzenethiol-6-yloxy)acetic acid, 1-Aza-.alpha.-tocopherol-6-yloxyl-acetic acid, 1-Aza-N-methyl-.alpha.-tocopherol-6-yloxyl-acetic acid or 2,5,7,8-Tetramethyl-2R-(4,8,12-trimethyl-3,7,11 E:Z tridecatrien)choman-6-yloxy)acetic acid.

8. The method of claim 7, wherein the chroman ring derivative compound is α-TEA or an α-TEA derivative wherein the isopernyl side chain is substituted for a phytyl side chain.

9. The method of claim 8, wherein the chroman ring derivative derivative compound is α-TEA.

10. The method of claim 1, wherein the cancer patient comprises a bladder, blood, bone, brain, breast, colon, esophagial, gastrointestinal, gum, head, kidney, liver, lung, nasopharynx, neck, ovary, prostate, skin, stomach, testicular, tongue, or uterine cancer.

11. The method of claim 10, wherein the cancer patient comprises a skin, breast or prostate cancer.

12.-15. (canceled)

16. The method of claim 1, wherein the cancer does not comprise a consitutivly active Akt kinase.

17. A method for treating a cancer patient comprising:
(i) obtaining or having a sample from the patient comprising proteins or nucleic acids from a cancer cell;
(ii) determining whether the cancer cell expresses a Arg, p73, NOXA or FOXO1 gene; and
(iii) treating the patient with an effective amount of a chroman ring derivative compound or another anti cancer therapy depending upon whether the cancer cell expresses a Arg, p73, NOXA or FOXO1 gene.

18. The method according to claim 17, wherein determining whether the cancer cell expresses a Arg, p73, NOXA or FOXO1 gene comprises determining whether the cancer cell express two or more of said genes.

19.-42. (canceled)

43. A method for treating a patient with a hyperproliferative disease comprising administering to the patient an effective amount of a chroman ring compound in combination with an Akt or PI3 kinase inhibitor.

44. The method of claim 43, wherein the chroman ring derivative compound is an alpha, beta, gamma or delta tocopherol or tocotrienol.

45.-48. (canceled)

49. The method of claim 43, wherein the chroman ring compound is administered in combination with a Akt or PI3 kinase inhibitor.

50.-57. (canceled)
